(19) United States
(12) Patent Application Publication
(10) Pub. No.: US 2026/0104399 A1
(43) Pub. Date: Apr. 16, 2026

(54) A PROCESS FOR ASSESSING BIODEGRADABILITY OF COMPLEX ORGANIC MIXTURES

(71) Applicant: ABOCA S.P.A. SOCIETÀ AGRICOLA, Sansepolcro (IT)

(72) Inventors: Luisa MATTOLI, Sansepolcro (IT); Mattia GIANNI, Urbania (IT); Giacomo PROIETTI, Pierantonio (IT); Emiliano GIOVAGNONI, Sansepolcro (IT); Valentino MERCATI, Sansepolcro (IT)

(73) Assignee: ABOCA S.P.A. SOCIETÀ AGRICOLA, Sansepolcro (IT)

(21) Appl. No.: 19/102,728

(22) PCT Filed: Aug. 4, 2023
(86) PCT No.: PCT/IB2023/057905

§ 371 Date: Feb. 10, 2025

(30) Foreign Application Priority Data
Aug. 11, 2022 (IT) 102022000017244

(51) Int. Cl. G01N 30/72; G01N 30/02; G01N 30/86; G01N 30/88; G16C 20/30; G16C 20/70

(52) U.S. Cl. CPC G01N 30/7233; G01N 30/86; G01N 30/88; G16C 20/30; G16C 20/70; G01N 2030/027; G01N 2030/884

(57) ABSTRACT

The present invention relates to a process for assessing the biodegradability of complex organic mixtures such as pharmaceutical compositions allowing the understanding of the fate of complex products that cannot be assessed by the standard Ready Biodegradability Tests currently available (RBTs)

The present invention relates to a process for assessing the biodegradability of complex organic mixtures such as pharmaceutical compositions allowing the understanding of the fate of complex products that cannot be assessed by the standard Ready Biodegradability Tests currently available (RBTs)

### STATE OF THE ART

Biodegradation is the process by which organic substances are decomposed by microorganisms into the simplest natural building blocks (e.g. CO₂, H₂O, NH₃) that can be integrated into the natural biogeochemical cycles. Anthropic and industrial activities have led to the emergence of a series of new polluting compounds and their release into the environment is responsible of a number of adverse effects, stimulating the development of a number of protocols to attempt their removal.

The evaluation of the biodegradability of chemicals is one of the main issues in environmental risk assessment. Biodegradability tests are designed to evaluate, in batch condition a chemical substance as the sole carbon source for the microfauna survival.

The Ready Biodegradation Tests (RBT) are at the basis of the integrated testing strategy on pure substances biodegradation. They are a series of tests (from n°301A to 301F and n° 310) proposed by Organization for Economic Cooperation and Development (OECD). Microorganisms and the tested substance are usually incubated in a buffered pH7 medium containing N, P, and trace element (named as “mineral medium”). The kinetic of biodegradation is monitored during at least 28 days by the evaluation of metabolic parameters such as oxygen consumption, carbon dioxide production, or dissolved organic carbon consumption. RBT measures ultimate biodegradability, or complete biodegradation and a chemical can be classified as readily biodegradable if has passed one of the RBTs (OECD, Revised Introduction to the OECD Guidelines for Testing of Chemicals, Section 3, OECD, 2006).

Specific chemical analysis can be used to assess primary biodegradation of the tested substance and to determine the concentration of any new formed intermediate.

This additional evaluation is mandatory only in the MITI method (301 C), but it is optional for all other RBTs (OECD, Revised Introduction to the OECD Guidelines for Testing of Chemicals, Section 3, OECD, 2006, OECD, Test No. 301: Ready Biodegradability, 1992.; OECD, «Test No. 310» Ready Biodegradability—CO₂ in sealed vessels (Headspace Test), 2014.).

With the term, primary biodegradation is indicated the structural modification of a substance, caused by a biological event, which results in the loss of a specific property of that substance.

It can be calculated from supplemental chemical analysis for parent compounds made at the beginning and end of the tests (OECD 301, 310) (REGULATION (EC) No 440/2008 of 30 May 2008).

A not-readily biodegradable substance is considered persistent unless its environmental degradability is proven in more expensive and complex simulation tests, (tests method N° 303, 306, 307, 308, and 309, OECD, 1992c, d, f, g, h). The type of simulation tests to be performed depends on the potential receptor environments that are causing concern (wastewater treatment plant, surface water, sediment, soil). Simulation tests are designed to evaluate the long-term chemical behaviour in the environment. However, these tests are limited to a very small number of pure substances because they are expensive, technically sophisticated, and time-consuming (e.g. need the use of several different tests and the use of ¹⁴C-labeled chemicals) (N. Nyholm, A. et al comparative study of test methods for assessment of the biodegradability of chemicals in seawater—Screening tests and simulation tests, Ecotoxicol. Environ. Saf., 1992, 23, 173190; J. F. Ericson, Evaluation of the OECD 314B Activated Sludge Die-Away Test for Assessing the Biodegradation of Pharmaceuticals, Environ. Sci. Technol., 2010, 44, 375-381; F. Brillet, A. Maul, M. J. Durand and T. Gérald, From laboratory to environmental conditions: a new approach for chemical's biodegradability assessment, Environ. Sci. Pollut. Res. 2016, 23, 18684-18693).

In silico models have been developed (F. Pizzo, A. Lombardo, A. Manganaro and E. Benfenati, In silico models for predicting ready biodegradability under REACH: a comparative study, Sci. Total Environ., 2013, 463-464, 161-168.) and are available under REACH regulations.

However, the prediction of overall persistence with multimedia fate models remains limited to existing data and may be not adapted to assess environmental persistence as a function of both chemical intrinsic properties and environmental conditions.

The currently adopted tests for biodegradability even if are cheap and easy to be performed present some limitations. As an example, they are conducted in standardized conditions, which do not reflect highly variable environmental situations like seasonality. Furthermore, even if they allow risk assessment by conservative models, tests most chemicals at concentrations that are unlikely occurring in the environment.

Some additional criticism can be referred to the microbial inoculum used for the biodegradation tests. Before being used, inoculum sources need to be either washed (to limit carbon contamination other than that from the tested substance) or acclimated (e.g for the 301C test,).

In addition, the final output may be influenced by the total cell density, the diversity of species, the origin and history of the inoculum sample, the ratio between food and biomass, the duration of the evaluation period that, in the standard experiment, has been defined at 28 days.

A further limitation of these tests is that usually they do not take in consideration the outcome of the biodegradation supposing that the investigated molecule is fully degraded into elemental bricks. Nowadays, the development of modern and reliable analytical techniques offers the possibility to investigate the biodegradability of a substance (or a mix of substances like in a pharmaceutical, nutraceutical or cosmetic formulation) in a more accurate manner both at qualitative and quantitative levels, offering an interesting and unprecedent complementary analysis at the currently used primary biodegradation studies.

The use of novel technologies enables in our opinion a more accurate and realistic evaluation of the environmental risks and impact, looking toward a more sustainable development.

Furthermore, Ready Biodegradability Tests and simulation tests are usually referred to pure chemicals, and only few cases of mixtures composed by structurally similar chemicals are reported. One example is that of petroleum and surfactants. It was reported that the kinetics observed for the biodegradation of the mixture and those of the isolated chemical entities are considerably different, with these latter in general faster than that of the mixture (European Chemicals Agency, 2017, Guidance on Information Requirements and Chemical Safety Assessment Chapter R.7b: Endpoint specific guidance Draft Version 4.0 January 2017.).

Clearly, moving from a reductionistic analytical investigation of a single molecule biodegradation to the holistic one that will take in consideration the biodegradability of a pool of chemical ingredients could represent a strong innovation and, potentially, a real improvement in the design of a new reliable sustainable development.

### SUMMARY OF THE INVENTION

The authors of the present invention carried out RBTs tests on complex mixtures of organic compounds and have also observed that the biodegradability of a mixture of compounds cannot be considered as the simple sum of the biodegradability of each component. In addition, the authors have observed that the currently available RBTs techniques are not informative on the kind of degradation undergone by complex mixtures and do not provide exhaustive information on the environmental impact of complex mixtures of organic compounds including pharmaceutical compositions or cosmetical compositions or food supplement compositions or medical device compositions or nutraceutical compositions.

The authors of the invention compared the biodegradation of compositions having a comparable medical effect but different components, such as two cough syrups and two compositions used for reflux disease and functional dispepsia.

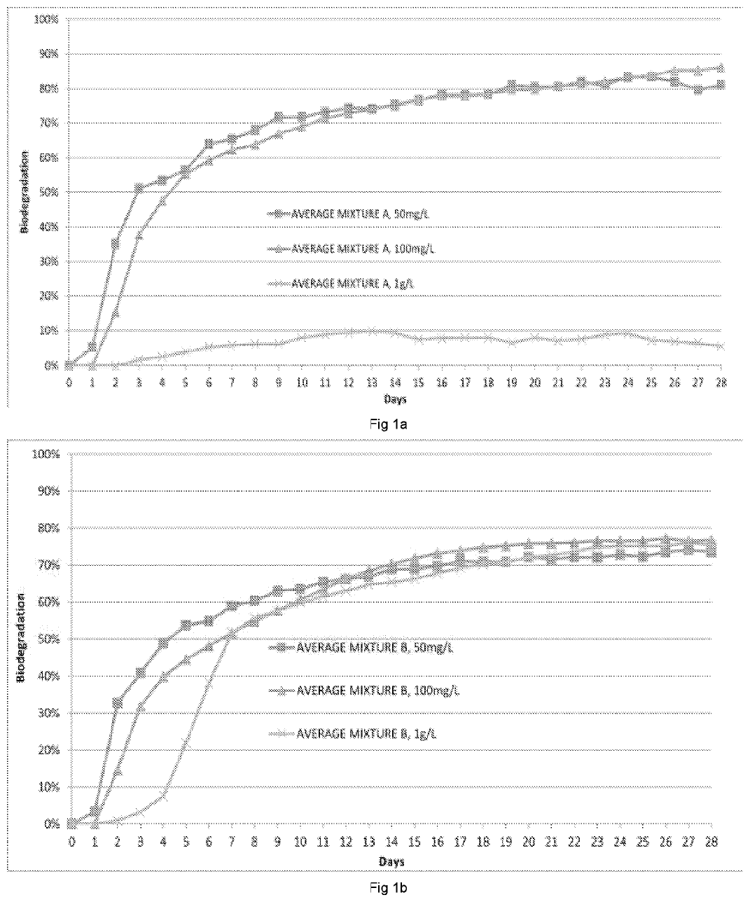

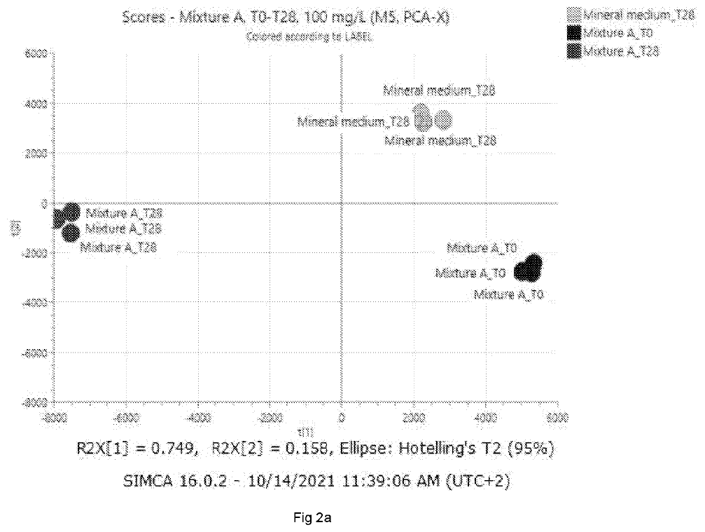

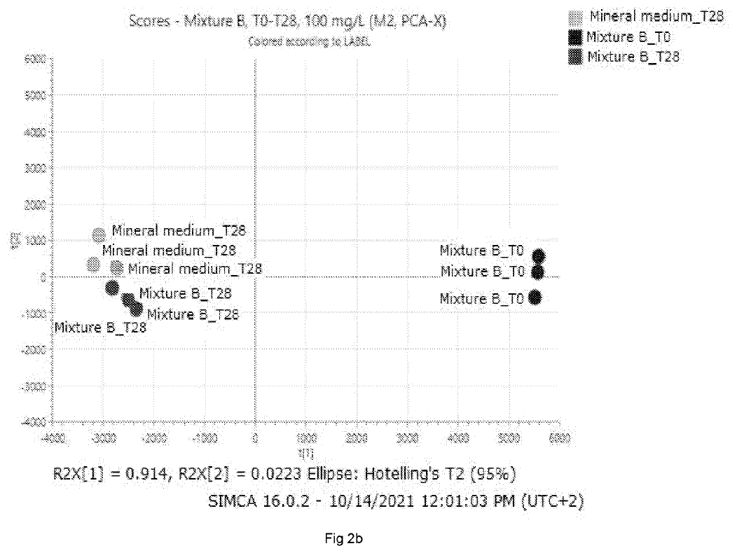

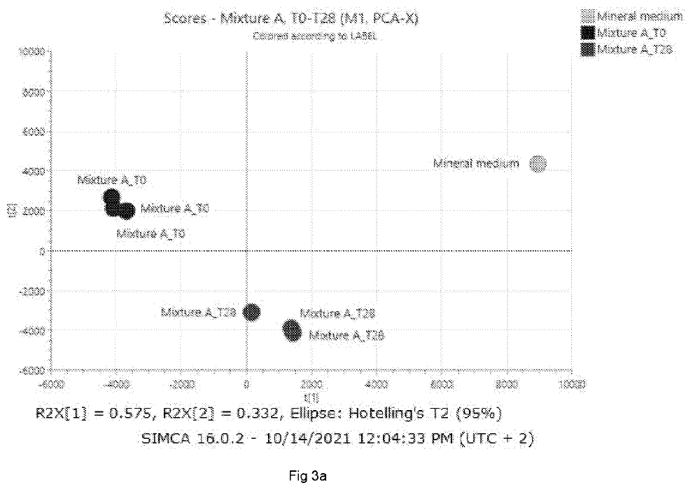

The authors have verified that, although the results provided by RBTs indicate the biodegradation of the cough syrups as substantially superimposable (see FIGS. 1 and 2 and table 2 experimental section) a more detailed analysis with the method provided in the present description and claims, allowed to verify that the degradation products were strongly different from one syrup tested to another. Also the tests carried out on the gastric compositions demonstrated that the method provided in the present invention provides additional and relevant information on the overall degradation of the composition tested.

No information on the mineralisation of the compositions tested was provided by the standard RBTs.

The authors of the invention have hance developed a method that provides additional relevant information on the mineralisation of the mixtures tested, thereby providing important information on the degradation products of said mixtures.

The method of the invention comprises RBTs standard steps, and additional steps that enables the user to assess a degree of similarity between the degraded mixture and the environment in which degradation is tested without the mixture (blank). The more the composition is similar to the blank the more the organic compounds of the mixture are mineralised. The method of the invention, hence, provides relevant information on the degree of mineralisation of mixtures of organic compounds.

Object of the invention is hence a method for the assessment of the biodegradability of mixtures of organic compounds comprising the following steps:
a) preparing at least one test flask or vessel containing a predetermined amount of a mixture of organic compounds of interest suspended in a suitable mineral medium together with an inoculum, wherein said inoculum is obtained from activated sludge; sewage effluents (unchlorinated); surface waters and soils; or from a mixture thereof and at least one blank flask or vessel containing only said mineral medium and said inoculum
b) acquiring the fingerprint by liquid chromatography coupled to mass spectrometry of a sample from each flask or vessel prepared in a) at T0, T0 being the day of the preparation a) wherein each sample is submitted to filtration in order to eliminate microfauna therefrom before acquiring said fingerprint
c) acquiring the fingerprint by liquid chromatography coupled to mass spectrometry of a sample from each test flask or vessel prepared in a) at Tn, n being any integer >0 and representing the number of the day after said preparation in a) wherein each sample is submitted to filtration in order to eliminate microfauna therefrom before acquiring said fingerprint
d) carrying out a multivariate statistical analysis on the data obtained in b) and c)
e) comparing the multivariate statistical analysis results obtained in d) for each test sample and for the blank sample and assessing the biodegradation of each test sample by evaluating the distance between the data obtained for the blank sample at Tn and for the test samples at least at T0 and at said Tn.

### Glossary

The term “mineral medium” has the meaning commonly used in the art for RBT standard tests (e.g. as in OECD, Revised Introduction to the OECD Guidelines for Testing of Chemicals, Section 3, OECD, 2006; OECD, Test No. 311: Anaerobic Biodegradability of Organic Compounds in Digested Sludge: by Measurement of Gas Production, OECD, 2006; OECD, Test No. 310: Ready Biodegradability—CO₂ in sealed vessels (Headspace Test), OECD, 2014; OECD, Test No. 301: Ready Biodegradability, 1992).
The term “mineralisation” has the meaning commonly used in the art and indicates the decomposition of the chemical compounds in organic matter, by which the nutrients in those compounds are released in soluble inorganic forms (minerals).
Pooled sample according to the present invention has the meaning commonly used in the art and defines a quality control sample comprising equal amounts of each of the tested samples (i.e. blank excluded).
T0 according to the present description is the day in which the test begins, i.e. the day in which the preparation of a) is made.
Tn according to the present description is any day after the day of T0, hence T1 is the first day after the day in which the preparation in a) has been made, T28 is the 28^{th} day after the day in which the preparation in a) has been made and so on.
Inoculum according to the present description is generally referred to as microfauna obtained from collection of activated sludge, sewage effluents (unchlorinated), surface waters and soils, or from a mixture thereof. Said microfauna is generally mainly composed by cercozoa, metazoans, ciliated protozoa, amoebas; sometimes tardigrades and filamentous bacterial can be present. In general, inoculum contents any species that can be commonly present in the microfauna communities of activated sludge, sewage effluents (unchlorinated), surface waters and soil
Test flask or vessel, according to the present description is a flask or vessel containing the mixture of organic components of interest, a suitable mineral medium and an inoculum, wherein said inoculum is obtained from activated sludge; sewage effluents (unchlorinated); surface waters and soils; or from a mixture thereof.
Blank flask or vessel, according to the present description is a flask or vessel containing a suitable mineral medium and an inoculum, wherein said inoculum is obtained from activated sludge; sewage effluents (unchlorinated); surface waters and soils; or from a mixture thereof.

The terms targeted methods, suspect screening and untargeted screening (or analysis) are used in the present description.

Depending on the level of pre-existing knowledge associated to the considered compounds of interest, three related methodological approaches can be used to stratify the chemical burden in samples from different sources, namely (i) targeted methods for known compounds, (ii) suspect screening for known unknowns and (iii) untargeted screening for unknown unknowns.

Suspects can potentially be further “converted” into targets by collecting comprehensive mass spectrometric reference data that enables unequivocal identification of the suspect compound (usually reliant on the availability of reference standard compounds). The remaining signals in the sample are generally termed “un-targets” or “unknown-unknowns”, for which no identity can be readily assigned, requiring further structural elucidation.

By definition “targets” are compounds of known chemical name and structure, for which quantitative targeted methods are available and a number of guidelines already exist to harmonize method performances assessment (e.g. Commission Decision 2002/657/EU for food). Targeted screening can be also conducted using high resolution instrumentation (e.g. quadrupole time of flight—qToF) which opens the door to simultaneous targeted, suspects and untargeted analyses.

“Suspects” are known compounds in terms of chemical name and structure which are expected (“suspected”) to be present in a sample. The typical approach applied in this case is suspect screening aiming to generate semi-quantitative data and contribute to better elucidate the composition of complex mixtures by simultaneously generating data for a wide range of compounds from each individual sample. In most cases, analytical standards are not readily available and therefore, relevant analytical methods are not validated and compound identities not definitive.

The qualitative annotation step refers to the assignment of a given compound identity to a signal detected by the suspect approach and relies on the presence of list of compounds or on the elaboration and implementation of reference libraries to match the generated experimental data with structural descriptors indexed from a list of a defined chemical compounds.

At last, “untargeted” analysis tries to detect “unknown unknowns” compounds without definition of any a priori criteria. Generally, sample preparation and data acquisition are similar between suspect screening and untargeted analysis whereas data analysis and data mining are different. Although highly challenging, these approaches represent the most promising strategy to advance our knowledge of complex mixtures.

### DETAILED DESCRIPTION OF THE DRAWINGS

FIG. 1 Biodegradation of the mixtures A and B (panels A and B respectively) at different concentration. All the data are reported as average of two or three replicates.

FIG. 2. PCA models for samples A and B at 100 mg/L (panels A and B respectively).

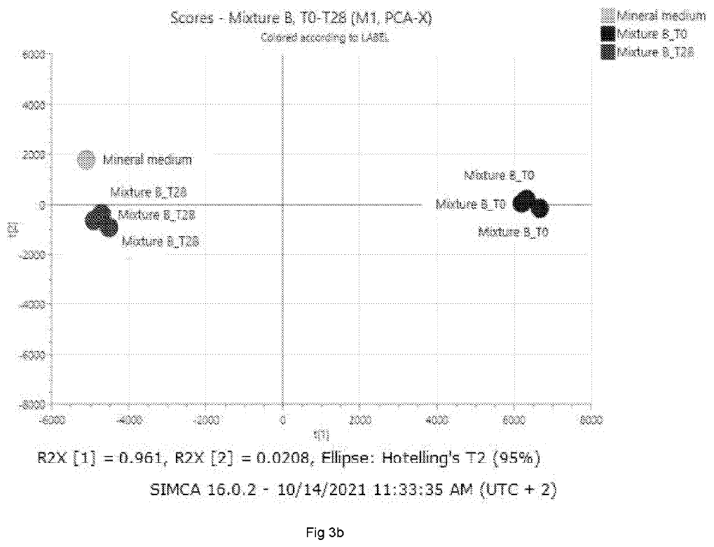

FIG. 3. PCA models for samples of mixtures A and B at 1000 mg/L (panels A and B respectively).

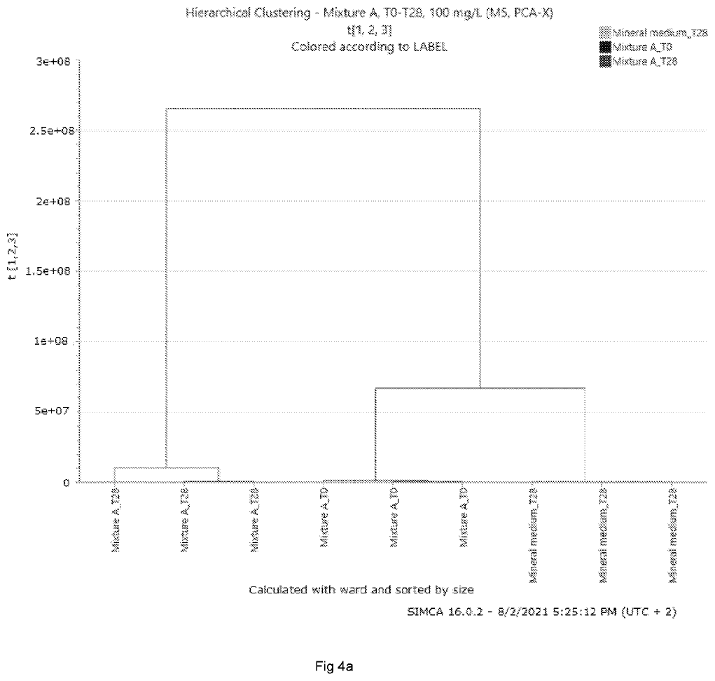

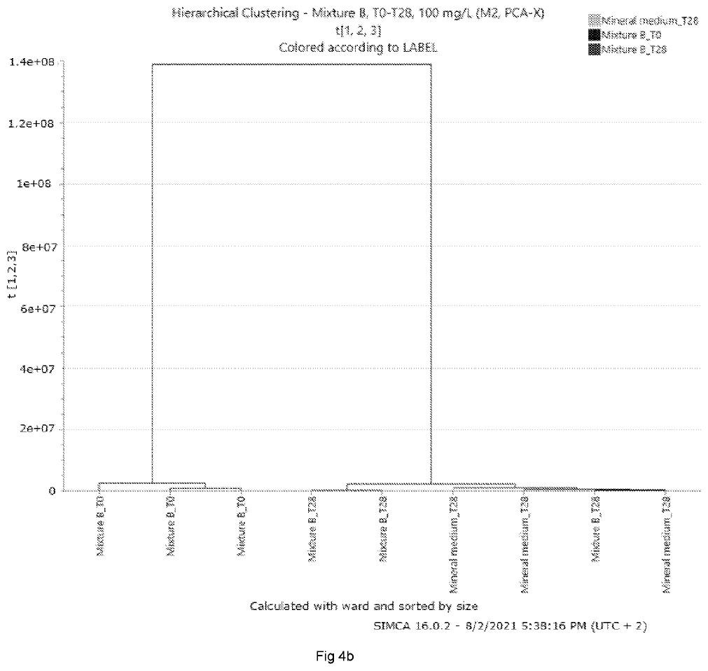

FIG. 4. Cluster analysis for samples of mixtures A and B at 100 mg/L (panels A and B respectively).

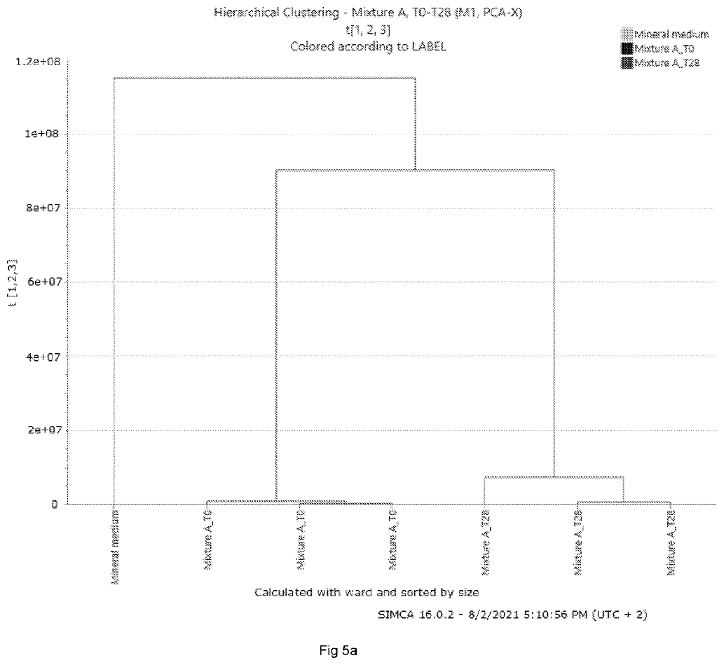

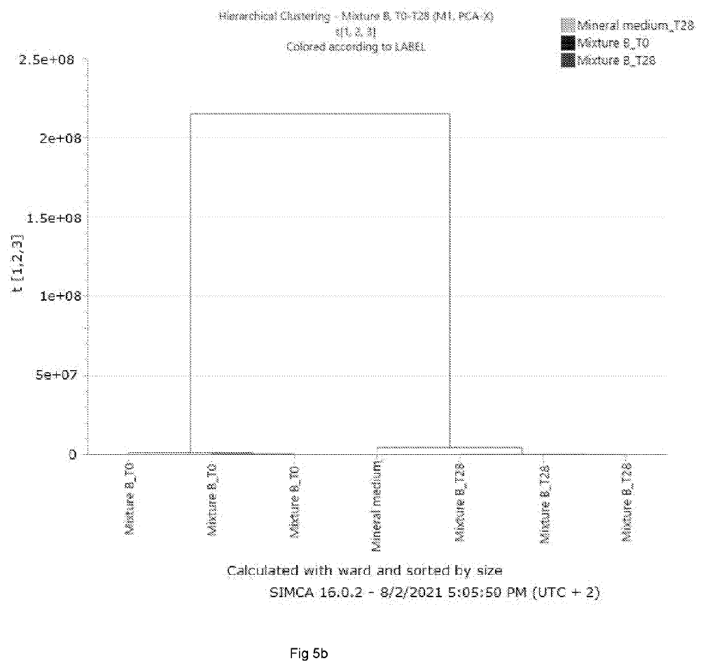

FIG. 5. Cluster analysis for samples of mixtures A and B at 1000 mg/L (panels A and B respectively).

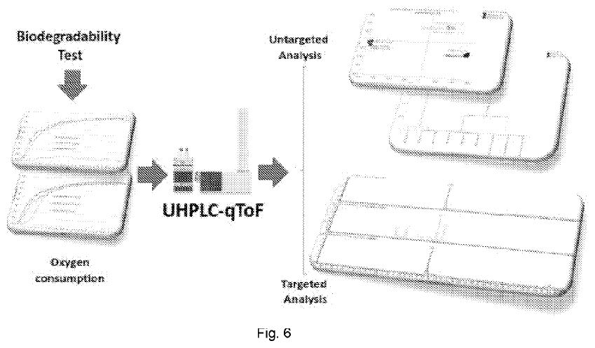

FIG. 6 Schematisation of the method of the invention.

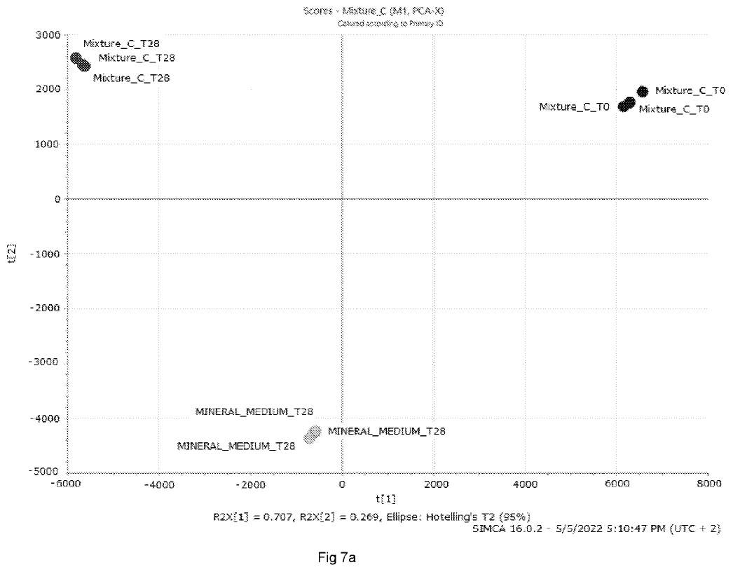

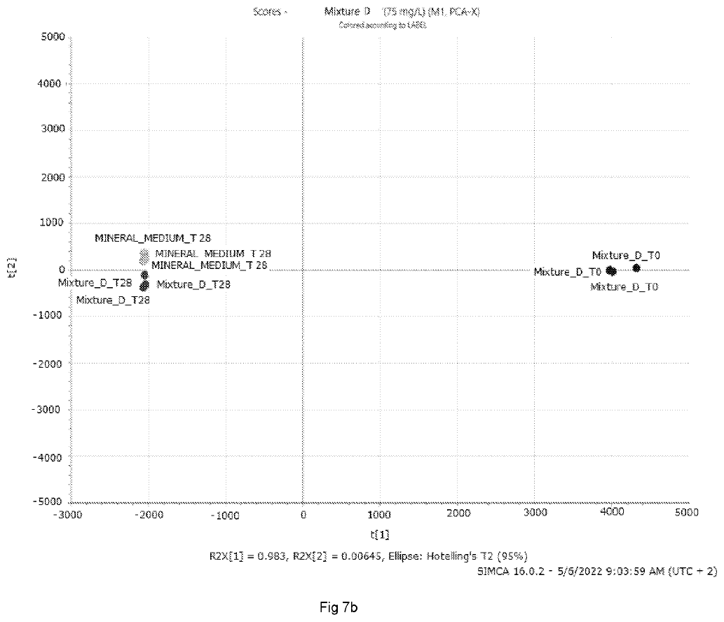

FIG. 7. PCA models for samples of mixtures C (50 mg/L) and D (75 mg/L), from the ready biodegradability test OECD 301F (panels A and B respectively).

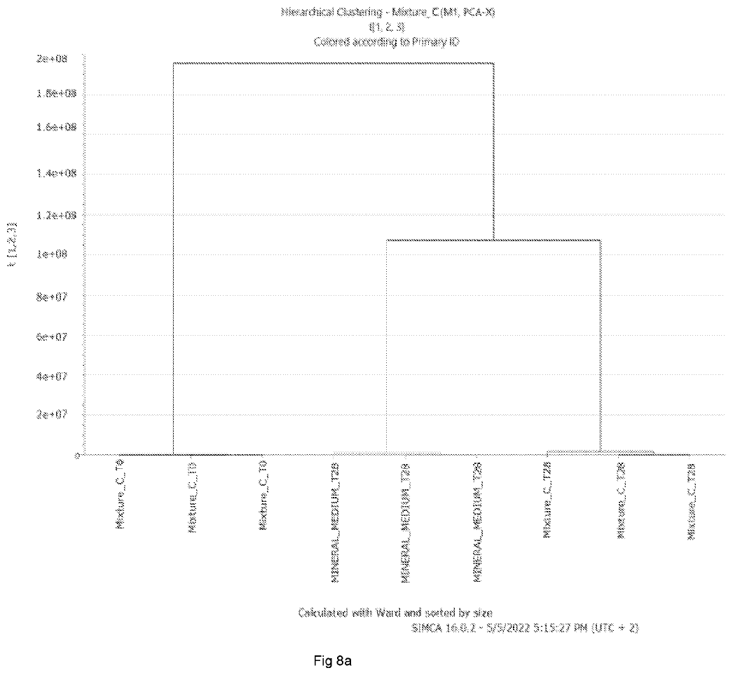

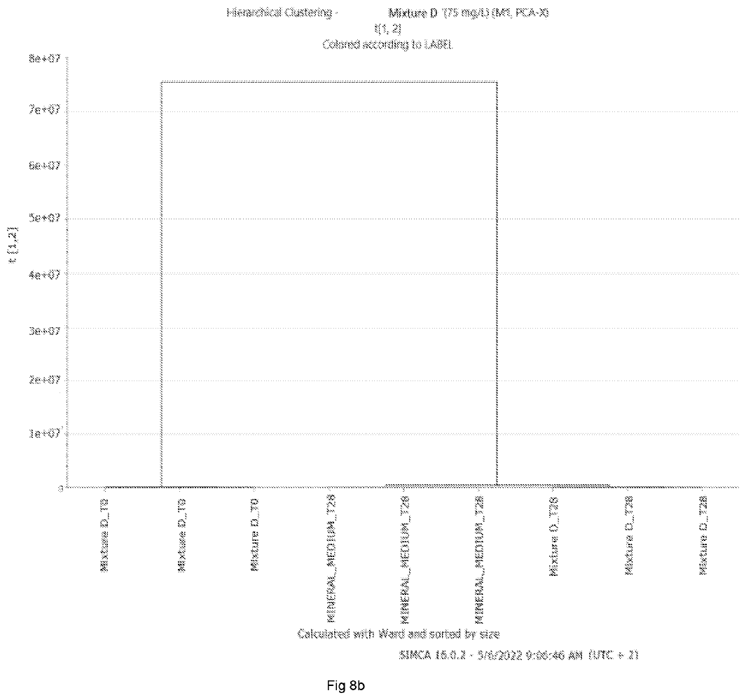

FIG. 8. Cluster Analysis for samples of mixtures C (50 mg/L) and D (75 mg/L), from the ready biodegradability test OECD 301F (panels A and B respectively).

### DETAILED DESCRIPTION OF THE INVENTION

The present invention provides for the first time a method for evaluating the biodegradability of complex mixtures of organic compounds.

The effect of the medical compositions in the form of complex mixtures of organic compounds, i.e. medical compositions such as medical compositions comprising plant extracts as active principles and/or comprising a number of organic compounds (wherein said number is of at least two but preferably at least 10, at least 100, at least 1000), on the environment is becoming more and more relevant and at present there are no described methods nor standardised protocols for the assessment of the biodegradability complex mixtures of organic compounds also in the form of pharmaceutical compositions.

The available methods, OECD Ready Biodegradability Tests (RBTs) (OECD, Revised Introduction to the OECD Guidelines for Testing of Chemicals, Section 3, OECD, 2006; OECD, Test No. 311: Anaerobic Biodegradability of Organic Compounds in Digested Sludge: by Measurement of Gas

Production, OECD, 2006; OECD, Test No. 310: Ready Biodegradability—CO2 in sealed vessels (Headspace Test), OECD, 2014; OECD, Test No. 301: Ready Biodegradability, 1992.) are not suitable to assess the biodegradability of complex mixtures of organic compounds such as pharmaceutical compositions, cosmetical compositions, and the like, and to not provide information on the level of mineralisation of said mixtures in standard biodegradability assays.

The authors of the present invention analysed by RBT OECD N°301F (OECD, Test No. 301: Ready Biodegradability, 1992; OECD, «Revised Introduction to the OECD Guidelines for Testing of Chemicals, Section 3, OECD» 2006) of four different compounds, the results obtained show that no information on the degradation output of the compositions is obtainable with said method. The authors hence developed a method that enables the skilled person to readily assess the level of mineralisation of the mixtures analysed.

The tested products are Mixtures A, B, C and D whose composition is summarised in Table 1 in the examples below.

Object of the present invention is method for the assessment of the biodegradability of mixtures of organic compounds comprising the following steps:
a) preparing at least one test flask or vessel containing a predetermined amount of a mixture of organic compounds of interest suspended in a suitable mineral medium together with an inoculum, wherein said inoculum is obtained from activated sludge; sewage effluents (unchlorinated); surface waters and soils; or from a mixture thereof and at least one blank flask or vessel containing only said mineral medium and said inoculum
b) acquiring the fingerprint by liquid chromatography coupled to mass spectrometry of a sample from each flask or vessel prepared in a) at T0, T0 being the day of the preparation a) wherein each sample is submitted to filtration in order to eliminate microfauna therefrom before acquiring said fingerprint
c) acquiring the fingerprint by liquid chromatography coupled to mass spectrometry of a sample from each test flask or vessel prepared in a) at Tn, n being any integer >0 and representing the number of the day after said preparation in a) wherein each sample is submitted to filtration in order to eliminate microfauna therefrom before acquiring said fingerprint
d) carrying out a multivariate statistical analysis on the data obtained in b) and c)
e) comparing the multivariate statistical analysis results obtained in d) for each test sample and for the blank sample and assessing the biodegradation of each test sample by evaluating the distance between the data obtained for the blank sample at Tn and for the test samples at least at T0 and at said Tn.

Furthermore, according to the invention step c) can be repeated at different Tn and said steps d) and e) are carried out for each fingerprint of the test sample and blank sample acquired at the same Tn for each value of n.

According to the present invention, a non-limiting example of mixture of organic compounds is represented by pharmaceutical compositions or cosmetical compositions or food supplement compositions or medical device compositions or nutraceutical compositions. According to one embodiment of the invention, said mixture of organic compounds comprises at least one plant extract or at least a fraction of a plant extract.

According to the present invention, the mineral medium is the one also described in the art for standard RBT test, and is normally prepared from stock solutions of appropriate concentrations of mineral components potassium and sodium phosphates plus ammonium chloride, calcium chloride, magnesium sulphate and iron (III) chloride.

Various stock solutions can be used and they can be mixed and diluted appropriately in order to prepare the suitable medium. The person skilled in the art can make reference to any OECD RBT protocol such as the OECD, Test No. 301: Ready Biodegradability, 1992 and the Revised Introduction to the OECD Guidelines for Testing of Chemicals, Section 3, OECD, 2006.

Various mineral media for bacteria growth are known in the art and are suitable for carrying out the present invention. The medium can be a basal minimal medium or a supplemented minimal medium (e.g. with growth factors or the like) depending on the inoculum.

In any case, the sole source of organic carbon in the medium in the method of the present invention is, as in RBT tests, provided by the mixture of organic compounds of interest, i.e. the mixture whose degradation is analysed by the method of the present invention.

According to the invention the inoculum is prepared following standard RBT protocols, such as, by way of example, OECD 301 (A to F) or similar.

The inoculum may be derived from a variety of sources: activated sludge; sewage effluents (unchlorinated); surface waters and soils; or from a mixture of these. By way of example, the activated sludge can be taken from a treatment plant or laboratory-scale unit receiving predominantly domestic sewage or from a mixture of sources.

The skilled person can readily follow, OECD 301 A-F and OECD 310 in order to prepare the inoculum. In one embodiment of the invention OECD 301 F can be followed, detailed protocol s for the preparation of a suitable inoculum can be found in OECD GUIDELINE FOR TESTING OF CHEMICALS Adopted by the Council on 17 Jul. 1992 pages 48 to 51.

Without limiting the invention, the inoculum can be prepared by collecting a fresh sample of activated sludge from the aeration tank of a sewage treatment plant or laboratory-scale unit treating predominantly domestic sewage and removing coarse particles if necessary and keeping the sludge aerobic thereafter. In case the sludge contains or is thought to contain inhibitors the sludge is preferably washed and resuspended for inoculation. Preferably the main microfauna components of the inoculum are determined prior use e.g. by optical microscopy. The inoculum comprises/consists of bacteria/microfauna isolated from the sources described above.

Alternatively, the inoculum can be derived from the secondary effluent of a treatment plant or laboratory-scale unit receiving predominantly domestic sewage and/or surface water, e.g. river, lake, pond or the like. If necessary, the inoculum can be concentrated.

According to the invention each mixture of interest will be suspended in a suitable amount of mineral medium together with the inoculum as described above. Preferably at least two flasks or vessels for each mixture of interest will be prepared in step a) of the method herein described and claimed, even more preferably the test will be carried out in triplicate and three flasks or vessels for each mixture of interest will be prepared in step a). Flask or vessels comprising the mixture of interest are also herein defined test flask or test vessel.

According to the invention, the method may further comprise a step f) of measuring oxygen consumption, and/or carbon dioxide production, and/or dissolved organic carbon consumption by means of calibrated continuous reader probe/s, or by making discontinuous readings by arranging an appropriate number of vessels for each scheduled measurement, from T0 to T28, T28 being the 28^{th} day after said preparation. Any measurement oxygen consumption, and/or carbon dioxide production, and/or dissolved organic carbon consumption by means of calibrated continuous reader probe/s, or by means of discontinuous readings by arranging an appropriate number of vessels for each scheduled measurement, can be carried out according to any RBT standard method as described in OECD GUIDELINE FOR TESTING OF CHEMICALS Adopted by the Council on 17 Jul. 1992 pages 48 to 51. Preferably, the measuring in f) is a manometric respirometry in which O₂ consumption and/or CO₂ production are measured in continuous. The consumption of oxygen can, by way of example, be determined either by measuring the quantity of oxygen (produced electrolytically) required to maintain constant gas volume in the respirometer flask, or from the change in volume or pressure (or a combination of the two) in the apparatus. Evolved carbon dioxide is absorbed in a solution of potassium hydroxide or another suitable absorbent.

The amount of oxygen taken up by the microbial population during biodegradation of the test mixture (corrected for uptake by blank inoculum, run in parallel) can be expressed as a percentage of ThOD, or COD.

The method of the invention, in fact, provides, in step f) the necessary data for performing an RBT standard test.

According to the invention, the test samples collected in c) are preferably organised in a randomised sample queue prior to liquid chromatography coupled to mass spectrometry acquisition and said sample queue also comprises one or more pooled sample and one or more blank sample. Analytical blank and pooled samples can be acquired at the beginning, in the middle of and at the end of the sample sequence.

Although the method may be carried out with acquisition of fingerprint of the test and blank sample at a single Tn, acquisition of fingerprint of test and blank samples can be carried out in different days after the preparation a), hence at different values of n.

In this case the analysis of step d) and the comparison of step e) is carried out for each blank and test sample for which the fingerprint was acquired on the same day (same Tn).

Tn, in the present description and claims refers to the day in which the sample is collected and the fingerprint acquired.

As also defined in the glossary, a pooled sample according to the present invention is a quality control sample comprising equal amounts of each of the tested samples (i.e. blank sample/s excluded).

Each tested sample is submitted to filtration in order to eliminate the microfauna from the sample before acquiring said fingerprints [in b) and c)] This stops the biodegradation at the desired time moment Tn. This filtration can be carried out with any filter known by the skilled person to be suitable for cleaning a liquid sample from the microfauna it contains. A non-limiting example of suitable filters is represented by filters with pores of 0.45 mm or less, preferably ≤than 0.25 mm, such as 0.22 mm or less.

Liquid chromatography coupled to mass spectrometry can be carried out with any standard procedure known by the skilled person. Preferably, the liquid chromatography coupled to mass spectrometry of the method is carried out by UHPLC-qToF, i.e. a UHPLC/q-ToF-MS (ultra-high performance liquid chromatography-quadrupole time-of-flight mass spectrometry) is carried out. Commercially available reagents can be used.

According to the invention the multivariate statistical analysis is carried out by processing the data obtained from the liquid chromatography coupled to mass spectrometry and by analysing the complex fingerprint obtained therefrom.

Preferably an unsupervised untargeted analysis is carried out, therefore the algorithms used reads patterns from untagged data by an expert and compounds from fingerprints are not identified.

Multivariate data analysis (MVDA) facilitates the understanding and interpretation of fingerprints data by providing a holistic view of the association between compounds and corresponding metabolites.

The multivariate statistical tests are many. In particular, these can be divided into:
unsupervised methods such as Principal Component Analysis (PCA), Hierarchical Clustering Analysis (HCA)-Dendrogram, Heatmap
supervised methods such as Partial Least Squares (PLS), Partial Least Squares-Discriminant Analysis (PLS-DA), Orthogonal Partial Least Squares (OPLS), Orthogonal Partial Least Squares-Discriminant Analysis (OPLS-DA),

Among unsupervised algorithms PCA is one of the most widely used multivariate technique for exploratory analysis (Worley B, Powers R, “Multivariate Analysis in Metabolomics.” Current Metabolomics 1 (2013): 92-107). If we have a high-dimensional dataset, such as dozens or hundreds of compounds or peaks for each sample, we may wish to find combinations that best explain the total variation in the original dataset, displaying “natural” trends or patterns and clusters between the samples according to the experimental conditions. PCA is one of the most powerful methods to perform this type of dimension reduction. Although the number of PCs is equals to the number of variables, only a limited number of PCs are interpreted. Moreover, if the first few PCs can explain a large proportion of variation in the data, we can visualize the data using a 2-dimensional or 3-dimensional plot (called scores or loading) (Joliffe, Ian. Principal Component Analysis. John Wiley & Sons, 2005).

As an unsupervised method, PCA is commonly exploited in metabolomics studies to highlight experimental differences for samples grouping.

Clustering analysis aims at identifying how many groups are presents in the original dataset. All clustering algorithms group the observations, such that the samples in the same group or cluster are more similar to each other than to samples in other groups. Hierarchical clustering creates a hierarchy and uses a dendrogram to represent the hierarchical structure, or in other words the clusters are organized as a hierarchical tree.

The data can be processed by carrying out a peak peaking and peak alignment setting a suitable mass tolerance, retention time tolerance and signal filtering threshold.

According to the invention confidence level can be set between X AND Y %, preferably at 95%. Goodness of the fit can be evaluated by R²X algorithm and goodness of prediction can be evaluated by Q² algorithm.

The observation diagnostic can be carried out by Hotelling T² and DModX tests.

In particular, Mass tolerance can be set at 10 ppm, Retention time tolerance can be set at 10 seconds, Signal Filtering Threshold can be set at 1000 counts. Mass tolerance can also be set at 5 ppm.

Variables with a frequency of missing value and coefficient of variation more than 20% can be eliminated and data imputation can be performed. Additionally, a filter to eliminate those variables present in the analytical blank samples can be applied.

Median of the replicates can be applied to remove the effect of the random noise, while data normalization can be performed with PQN (Probabilistic Quotient Normalization) on reference pooled samples.

The processed data can be saved in CSV format and the data matrix in CSV format can been reworked by the program SIMCA (Version 16.0.2.10561, Jan. 22, 2020). Subsequently, data matrix can be mean centered and Pareto scaling can be applied prior to perform data analysis.

The processed data providing the complex fingerprint of the sample can be then analysed by PCA (Principal Component Analysis) and/or by Hierarchical Cluster Analysis.

Confidence level for both analysis is preferably about 95%.

Through the steps beforementioned a clean matrix of data can be hence used to build the statistical model, after passing the classical test as R², Q², Hotelling T², dMoDX. Covariance matrix and standardized principal component score were selected for principal component analysis (PCA) computation by means of the unsupervised approach. By means of CA, after applying the method of Ward for the formation of the hierarchical cluster, the distance between the groups was determined using the Euclidean distance and the results of the analysis of the clusters are presented as dendrogram.

Step e) of the method of the invention is the step providing the advantageous information on the mineralisation of the mixtures of interest. Indeed, carrying out PCA and/or HCA on the samples after a period of time after the preparation in a) of the test samples has lapsed (e.g. 28 days, T28, in line with the RBT standards) and/or at earlier stages (one or more other day Tn) and comparison with the PCA and/or HCA results for the blank sample, allow the user of the method to assess whether biodegradation has occurred or whether no biodegradation or uncomplete biodegradation have occurred, confirming or not the results of the RBT when also the RBT step (step f) is carried out. In fact, both PCA as well as HCA allow to visualise the distance between the test sample and the blank sample, in terms of dots or in terms of clusters.

In particular, when the multivariate statistical analysis is carried out by PCA the user of the method will assess, in step e), occurred biodegradation when the PCA results of the test sample at Tn are near the 2D space of the PCA results of the blank sample at the same Tn, and non-occurred biodegradation or only partial biodegradation when the PCA results of the test sample at Tn are not near the 2D space of the PCA results of the blank sample at the same Tn the test sample.

According to the present description, PCA results are considered “near” when the dots of both the test sample and of the blank sample, are within a rectangle of 2×3 meshes of the PCA grid (2 on the X axis and 3 on the Y axis), preferably of 2×2 meshes of the PCA grid, more preferably within a rectangle of 1×1 meshes of the PCA grid, even more preferably in the same mesh of the PCA grid

A clear example of “near” dots, i.e. biodegraded mixture of interest can be seen in FIGS. 2b, 3b and 7b and of “not near” dots are in FIGS. 2a, 3a and 7a.

The changes are considered much more relevant if the distances between the samples are observed in the X-axis, as this describes the PC1 (PC1 is the first Principal Component of the PCA) which contains the largest percentage of variance in the model. Distances along the Y axis, which describes PC2 (PC2 is the second Principal Component of the PCA), will express smaller differences between samples, as PC2 describes a lower percentage of variance.

When the multivariate statistical analysis is carried out by HCA the user of the method will assess, in step e) occurred biodegradation when the HCA results of the test sample at Tn form a cluster with the HCA results of the blank sample at the same Tn, and non-occurred biodegradation or only partial biodegradation when the HCA results of the test sample at Tn do not form a cluster with the HCA results of the blank sample at the same Tn.

Generally, in both analyses, the T0 test sample remains isolated from the other samples. However, when the test sample at Tn is close to the sample at T0 in PCA and forms a cluster with the sample at T0 in HCA, it means that biodegradation has not yet occurred.

In a preferred embodiment one of said Tn is T28.

If desired, and in particular when no biodegradation or uncomplete degradation has been assessed in e) targeted qualitative analysis of one or more of the test samples in order to identify the non-degraded components or the complex degradation intermediates in the sample.

According to the invention, the method can be advantageously applied to mixtures of organic compounds such as pharmaceutical compositions, cosmetical compositions, food supplement compositions, medical device compositions or a nutraceutical composition. The medical device composition is a composition according to any one of the classes described in Directive 2017/745/EEC on medical devices that medical devices that are composed of substances or of combination of substances that are absorbed by or locally dispersed in the human body.

In any part of the present description and claims the term comprising can be substituted by the term consisting of.

Examples are reported below which have the purpose of better illustrating the embodiments disclosed in the present description, such examples are in no way to be considered as a limitation of the previous description and the subsequent claims.

### EXAMPLES

TABLE 1
Tested Mixtures

| | Mixture D_“natural ingredients” |
|---|---|
| Mixture A_“Bromhexine based” | |
| Bromhexine, Malitol, Sucralose, | Honey* |
| Preservative and Flavouring | “Polyresin” composed by: |
| | Resins, Polysaccharides and Flavonoids |
| | from Grindelia*, Plantago* and |
| | Helichrysum* |
| | Sugar cane* |
| | Essential oil: lemon, sweet |
| | orange, myrtle; lemon natural |
| | flavouring; arabic gum; |
| | xanthan gum |
| | Water |
| Mixture C_“Omeprazole based” | |
| Each capsule contains 20 mg of | Each tablet contains: |
| Omeprazole. | Poliprotect ® a synergistic molecular |
| The other ingredients are: | complex of polysaccharides* (obtained |
| Capsule contents: sugar spheres | from Aloe vera, Malva Sylvestris and |
| (consisting of corn starch and sucrose), | Althaea officinalis), Limestone and |
| sodium lauryl sulfate, anhydrous | Nahcolite. |
| disodium phosphate, mannitol, | Titrated in polysaccharides |
| hypromellose 6 cP, macrogol 6000, talc, | (molecular weight >20,000 Dalton) ≥7.6% |
| polysorbate 80, titanium dioxide (E171) | (40 mg per tablet) |
| and copolymer of methacrylic acid- ethyl | Flavonoid fraction* (obtained from |
| acrylate (1:1). | Matricaria recutita and Glycyrrhiza glabra) |
| Capsule components: gelatin. Coloring | The other ingredients are: sugar cane*, |
| agents: quinoline yellow and titanium | arabic gum, natural mint flavor. |
| dioxide | * Ingredient from organic farming |

^{a} How is reported in the package leaflet.

Two commercially available cough syrups (one containing synthetic ingredients [A] and one containing only natural ingredients[B]) were chosen as real examples of largely distributed formulations of pharmaceutical ingredients. Their biodegradation has been evaluated and compared with that of the pure non-naturally occurring API (Active Pharmaceutical Ingredient) Bromhexine, using UHPLC-qToF “all-ions MS/MS” acquisition technique. The chemical compositions of mixtures A and B declared in the package insert of the analyzed pharmaceutical mixtures are summarized in Table 1 above.

The ultra-high performance liquid chromatography-quadrupole time-of-flight mass spectrometer (UHPLC-qToF) is a recent type of spectrometer characterized by an ultra-high performance chromatographic separation of the molecular species that has been successfully employed for its fast, high-resolution separations, high-resolution mass spectra and high sensitivity.

Even if high resolution mass spectrometry as well as other spectroscopic methods were previously reported for the investigation of the environmental impact produced by pharmaceuticals and cosmetic formulations this is the first UHPLC-qToF “all-ions MS/MS” approach to perform metabolites fingerprints by untargeted data treatment of pharmaceutical formulation submitted to a readily biodegradability test and targeted analyses for deeper investigation of the readily biodegradation output.

### Mixtures A and B

### Biodegradation Method

Glucose purum, Magnesium sulphate heptahydrate for analysis ACS Reag. Ph. Eur and Reag. USP, Sodium acetate anhydrous USP, Phosphoric acid 99%, Potassium phosphate dibasic 99+% anhydrous for analysis, Potassium phosphate monobasic 99% anhydrous for analysis, Iron (III) chloride hexahydrate pure, Sodium phosphate dibasic dihydrate 98+%, Calcium chloride anhydrous 99% pure, were purchased from Carlo Erba Reagents srl (Cornaredo, Milano, Italy). Ammonium chloride 99% puriss and Potassium hydroxide 85% puriss were purchased from Italchimica SPA (Pontecchio Polesine, Rovigo, Italy). Buffered Peptone water (ISO) was purchased from ThermoFisher.

Purified water was prepared by Arium Mini water purification system (from Sartorius Goettingen, Germany).

### Sample Preparation

The sample was treated according to the procedure reported in the respirometric manometric method n°301F (OECD). To be able to have the correct registration by the BOD sensor and enough material to perform the instrumental analysis planned few adjustments were done as reported in table S4.

TABLE S4
Experimental parameters for the respirometric test

| Sample concentration | | Volume of mineral media | Volume of the vessel |
|---|---|---|---|
| 50 | mg/L | 400 ml | 1 L |
| 100 | mg/L | 400 ml | 1 L |
| 1 | g/L | 100 ml | 1 L |

Sodium acetate was used as reference substance. Tests were done at the constant temperature of 22° C.

### Inoculum Preparation.

The inoculum was prepared collecting 5 samples of activated sludge and 5 samples of water river from different places and mixed in equivalent volume. The inoculum is oxygenated, stirred and fed with glucose, peptone, potassium phosphate dibasic and the values of redox potential, oxygen consumption and total dry matter are monitored daily. At the time of use, the dry substance was determined at 100° C. to measure the same quantity (30 mg/ml) in the vessels containing the test substance.

### Inoculum Composition

The inoculum was obtained by mixing active sludge from 8 different sectors and river water taken from two different rivers in equal parts. The assembled inoculum was oxygenated, stirred and fed with glucose, peptone and monopotassium orthophosphate. Oxygen, redox, and total suspended solids values were monitored daily.

Before the use of inoculum, the total dry matter is determined. The composition of the Microfauna was determined by optical microscope analysis and was composed by the following species: Aspidisca cicada, Zoothamnium, Rotiferi, Euglyphia.

### Instrumental Parameters

The biological oxygen demand was constantly monitored by means of BOD EVO Sensor System 6, being made up of six BOD EVO sensors, a six-position stirring base, dark glass bottles, subcaps for the absorption of carbon dioxide and magnetic stir bars. The BOD EVO sensor transmits wireless the data to the dedicated BODSoft™ software (VELP Scientifica, Usmate, Monza-Brianza, Italy). All the experiments were conducted under controlled temperature in a refrigerated thermostat provided with auto-tuning thermoregulation system and forced air circulation in order to have stability and homogeneity of the internal temperature (VELP Scientifica, Usmate, MonzaBrianza, Italy). The temperature of the thermostat was monitored by means of a datalogger with external probe with a range of temperature from −40 to +80° C. (XS Instruments, Carpi, Modena, Italy).

### UHPLC ESI-qToF Methods

All solvents were of high purity analytical grade and were used without further purification. ULC/MS grade absolute methanol was purchased from Biosolve (Dieuze, France). Ultrahigh purified water was prepared in a PURELAB® Ultra water purification system (ELGA, UK). Formic acid 98%-100% for LCMS LiChropur® and dimethyl sulfoxide ≥99% were purchased from Sigma-Aldrich (St. Louis, MO). Mixture A was purchased (at a local pharmacy, batch n° 181092), mixture B was produced by Aboca (batch n° 19C2076); Bromhexine hydrochloride, sucralose, maltitol and ambroxol were purchased from SigmaAldrich (St. Louis, MO). The high-purity reference standards, used both for the construction of the in-house database and for the construction of the calibration curves, were purchased from Extrasynthese (Genay, France), Sigma-Aldrich (St. Louis, MO), PhytoLab GmbH & Co. KG (Vestenbergsgreuth, Germany) and ChromaDex (Irvine, CA). High-purity reference standards stock solutions were prepared in methanol, water/methanol (80:20, v/v) or methanol/dimethyl sulfoxide (80:20, v/v) at 500 ppm. The working solutions were prepared by diluting appropriate volumes of the stock solutions with water/methanol (50:50, v/v). Internal standard sulfadimethoxine-d6 was purchased from Sigma-Aldrich (St. Louis, MO). Internal standard stock solution was prepared in methanol at 5 ppm. All the stock solutions were stored in glass vials at −80° C.

### Targeted Method

Sample preparation: The sample was filtered on a 0.20 μm Millipore cellulose acetate syringe filter and was used for the acquisition of the chromatographic profile without any dilution according to the following conditions.

Instrumental parameters. The instrumental platform used consisted of an UHPLC series 1290 coupled to a quadrupole time-of-flight (qToF) mass spectrometer series 6545 (Agilent Technologies, Santa Clara, CA) in high resolution (2 GHz). The UHPLC was endowed with a binary pump, an autosampler, a multicolumn thermostat, an isocratic pump and a solvent cabinet. The autosampler was maintained at 15° C. The analytical column was a Cortecs® C18 (100×2.1 mm, 1.6 μm) protected by a Cortecs® UPLC® C18 VanGuard™ pre-column (5×2.1 mm, 1.6 μm) both supplied by Waters (Milford, MA) and thermostated at 40° C. The analyses were performed in elution gradient using aqueous HCO₂H (0.1%, v/v) as mobile phase A and HCO₂H (0.1%, v/v in MeOH) as mobile phase B according to the following gradient table (Table S5) Below,

TABLE S5
UHPLC ESI-qToF Methods: Elution gradient

| Time (min) | A % | B % |
|---|---|---|
| 0.0 | 99 | 1 |
| 1.0 | 99 | 1 |
| 2.0 | 75 | 25 |
| 10.0 | 50 | 50 |
| 11.0 | 50 | 50 |
| 15.0 | 25 | 75 |
| 17.0 | 15 | 85 |
| 19.0 | 1 | 99 |
| 19.5 | 1 | 99 |
| 21.0 | 99 | 1 |
| 24.0 (post run) | 99 | 1 |

with a flow rate of 0.3 mL min⁻¹ The volume injected was 3 μL for the samples at 1000 mg/L, while was 10 μL for the samples at 100 mg/L.

The UHPLC system was coupled to a qToF mass spectrometer endowed with a Dual AJS ESI source operating in negative and positive ionization modes with a scan range from 50 m/z to 1700 m/z. The optimized instrument parameters are reported in Table S6 below.

TABLE S6
UHPLC ESI- qToF Methods: MS parameters

| Instrumental parameters | ESI− | ESI+ |
|---|---|---|
| Gas temperature (° C.) | 325 | 325 |
| Gas flow (L min−1) | 11 | 11 |
| Nebulizer (psig) | 35 | 35 |
| Sheat gas temperature (° C.) | 350 | 350 |
| Sheat gas flow (L min−1) | 12 | 12 |
| Vcap | 3500 | 3500 |
| Nozzle voltage (V) | 0 | 1000 |
| Fragmentor | 100 | 100 |
| Skimmer | 65 | 65 |
| Octopole RF Peak | 750 | 750 |

The construction of the in-house database was performed by acquiring each analytical standard reference compound in data dependent mode (Auto MS/MS, Agilent Technologies) using three different collision energy values (20, 30 and 40 eV) by N2. While the acquisition of the sample metabolite fingerprint under investigation was performed in All-Ions mode using 30 eV of collision energy value. In both the acquisition methods, a reference mass solution containing purine and hexakis (1H, 1H,3H-tetrafluoropropoxy) phosphazine was direct injected to the ESI source by the isocratic pump and ionized together with the sample solution for mass correction allowing to get accurate mass time-of-flight data. MassHunter software version B.07 (Agilent Technologies, Santa Clara, CA) was used for data acquisition and processing. Software. MassHunter software version B.07 (Agilent Technologies, Santa Clara, CA) was used for data acquisition and processing.

The exact mass of the various molecular species was calculated by means of Isotope distribution calculator a tool of MassHunter Data Analysis core, version 8.0.8208.0 (Agilent Technologies, Santa Clara, CA) Data Analysis. Elaborations were performed by means of “Qualitative analysis” program and by “Find-by-Formula” algorithm. The data file is loaded into “Qualitative analysis” then “Find-by-Formula” is run on the low channel against an MS/MS library. Find-by-Formula returns possible precursor formulas found as well as their product ions in the library. Using the list of product ions, “Qualitative analysis” extracts EICs from the high channel and aligns them with an EIC of the precursor.

A coelution score is calculated and compounds which pass the threshold (user-set at 70) are retained. “Qualitative analysis” returns the overall score to which contribute: the mass score, the isotope abundance score, the isotope spacing score and the retention time score the threshold was user-set at 70. EICs were taken by setting m/z extension to symmetric 2.5 ppm.

### Untargeted Method

Sample preparation: Samples were prepared as described for targeted UHPLC-qToF method.

### Multivariate Statistical Analysis

Data collection: Samples of the two syrups at T0 and T28 and the blank sample, consisting of mineral medium and inoculum, were organized into a randomized sample queue prior to UHPLC-qToF acquisition. The analytical blank and pooled samples were acquired at the beginning, in the middle of and at the end of the sample sequence. Each sample was acquired in triplicate.

Instrumental parameters were the same used for the targeted UHPLC-qToF method, positive ions for Mixture A and negative ions for Mixture B.

Data processing: Peak peaking and peak alignment were obtained using the software R [R version 3.5.2 (2018 Dec. 20), © 2018 The R Foundation for Statistical Computing]. Mass tolerance was set at 10 ppm and retention time tolerance was set at 10 seconds. Signal Filtering Threshold was set at 1000 counts.

Variables with a frequency of missing value and coefficient of variation more than 20% have been eliminated and data imputation was performed. Additionally, a filter to eliminate those variables present in the analytical blank samples was applied.

Median of the replicates was applied to remove the effect of the random noise, while data normalization was performed with PQN (Probabilistic Quotient Normalization) on reference pooled samples.

The processed data were saved in CSV format and the data matrix in CSV format has been reworked by the program SIMCA (Version 16.0.2.10561, Jan. 22, 2020). Later On, data matrix was mean centered and Pareto scaling was applied prior to perform data analysis.

Statistical Model performance evaluation was done and a confidence level of 95% was used.

The goodness of fit and the goodness of prediction were evaluated by means of R² X and Q², respectively (Table S7) below.

TABLE S7
Statistical Model performance evaluation by means of Q² and R²X.

| Sample | Q² | R²X |
|---|---|---|
| Mixture_A 100 mg/L | 0.849 | 0.937 |
| Mixture_B 100 mg/L | 0.818 | 0.953 |
| Mixture_A 1000 mg/L | 0.669 | 0.937 |
| Mixture_B 1000 mg/L | 0.864 | 0.989 |

The diagnostic tools Hotelling T² and DModX were calculated to be sure that the model is not deformed, therefore neither strong nor weak outliers were present. The models were robust, as the observations were below the calculated critical values and there were no outliers.

PCA analysis: After data processing, in order to evaluate the complex fingerprints obtained, multivariate analysis of the Principal Component (PCA) was used. The PCA model was built using the first two principal components providing a 2D model (Confidence level 95%).

Cluster Analysis: Cluster analysis was performed according to an unsupervised approach using a hierarchical algorithm (Confidence level 95%). Distance indices were determined using Ward's linkage method (J. H. Ward, Hierarchical Grouping to Optimize an Objective Function, J. Am. Stat. Assoc., 1953, 58, 236-244) and Euclidean distance.

### Semiquantitative Determination.

### —Relative Ratio (RR) Calculation.

The Relative Ratio (RR) is calculated by the ratio between the area counts of the compound and the area count of the internal standard (sulfadimethoxine-d6).

### —Calculation of the Percentage of Reduction

The percentage of reduction of a certain compound is the ratio between the RR at T28 and the RR at T0 expressed as a percentage.

### Results and Discussion

The UHPLC chromatographic conditions were optimized to obtain the best ionization along with the elution of all the species of interest within 21 minutes. Pure water and methanol both acidified with formic acid were used as mobile phase, composed according a linear gradient suitable for eluting hydrophilic and lipophilic compounds in a time frame compatible with a UHPLC analysis.

Through UHPLC-qToF “all-ions MS/MS” (S. Naz, H. Gallart-Ayala, S. N. Reinke, C. Mathon, R. Blankley, R. Chaleckis, C. E. Wheelock; Development of a Liquid Chromatography-High Resolution Mass Spectrometry Metabolomics Method with High Specificity for Metabolite Identification Using All Ion Fragmentation Acquisition. Analytical Chemistry 2017; 89:7933-7942) acquisition technique high resolution mass spectrometry (HRMS) data can be acquired using different conditions: (1) with a low value of collision energy (2) with a high energy value. The low energy spectra predominantly show just the molecular (or precursor) ions for the compounds and the high-energy spectra provide the precursors plus their fragment ions.

With All Ions MS/MS data, can be confidently carry out a targeted qualitative screening analysis with structure elucidation and identification of fragmentation patterns of the compounds presents in complex products also during the biodegradability studies.

Without resorting to structural identification, the large number of signals obtained from UHPLC-qToF analyses can be used to perform untargeted studies. Untargeted analysis gives information about the physical-chemical changes in samples during RBT, by observing the route of their grouping in statistical models by Principal Component Analysis (PCA) and cluster analysis (CA).

The respirometric-manometric test n° 301F (according to OECD guidelines OECD, Revised Introduction to the OECD Guidelines for Testing of Chemicals, Section 3, OECD, 2006) was chosen as method for the biodegradability evaluation.

It was performed at two concentrations required by the OECD301F method (50 mg/L and 100 mg/L) and, in addition, we tested an out-of-range concentration (1000 mg/L) aiming to a better identification of all the metabolites formed during the test. Samples were analysed at the beginning of the ready biodegradation test and after 28 days by means of the untargeted and targeted approaches.

Performing the RBT at a concentration of 50 mg/L mixture A passed the ten-days window criteria and after 28 days of incubation reached the 81% of biodegradation while mixture B passed the ten-days window criteria and reached the 74% of biodegradation at 28^{th} day. The curves of biodegradation are reported in FIG. 1. The test was repeated at the concentrations of 100 mg/L and 1000 mg/L. The results evidenced that the mixture A (100 mg/L BD28 days 86%) was again readily biodegradable at 100 mg/L but not at 1000 mg/L (1000 mg/L BD28 days 6%), while the mixture B was readily biodegradable in both the conditions (100 mg/L: BD28 days 77%, 1000 mg/L BD28: days 75%). These results are summarized in Table 2 as well as in FIG. 1.

TABLE 2
Results of the Ready Biodegradability test
(OECD 301F) of Mixture A and Mixture B

| Mixture | concentration | | 10 days criteria¹ | BD at 28^{th} day |
|---|---|---|---|---|
| A | 50 | mg/L | Passed | 81% |
| | 100 | mg/L | Passed | 86% |
| | 1000 | mg/L | Not Passed | 6% |
| B | 50 | mg/L | Passed | 74% |
| | 100 | mg/L | Passed | 77% |
| | 1000 | mg/L | Passed | 75% |

¹The pass levels for ready biodegradability are 60% of ThOD removed for respirometric methods. These pass values have to be reached in a 10-d window within the 28-d period of the test. The 10-d window begins when the degree of biodegradation has reached 10% ThOD and must end before day 28 of the test. Chemicals which reach the pass levels after the 28-d period are not deemed to be readily biodegradable.

Even if the 1000 mg/L is not a concentration considered in the validated method it is interesting to observe the different behaviours of the analysed samples. In the case of mixture A the microfauna of the inoculum seems to be unable to adequately activate the biodegradation, probably because it suffers of the experimental conditions. In our opinion, this can be ascribed to the high concentration of both, the substrate and/or some initially formed metabolites. A similar effect was not observed in the case of mixture B, indicating that different mixtures can exploit a different dose-dependent impact on the microenvironment of the experiment. In order to investigate better this phenomenon, after 28 days under the RBT conditions mixtures A at 50 mg/L, 100 mg/L and 1000 mg/L, respectively, were subjected to a spectrometric semiquantitative evaluation of the residual main components: sucralose 1, maltitol 2 and Bromhexine 3 (see below and Table S1).

TABLE S1
Semi quantitative evaluation of sucralose 1, malitol 2 and Bromhexine
3 by Relative Ratio RR estimation and calculation of the reduction
percentage. RR is the ratio between the area counts of the compound
and the area count of the internal standard (sulfadimethoxine-d6).
The level of reduction at the end of the experiment is the ratio
between the RR at T20 and the RR at T0 as a percentage.

| Concentration | | Sucralose (1) | Maltitol (2) | Bromhexine (3) |
|---|---|---|---|---|
| 50 | mg/L | RR at T0 0.049 | RR at T0 2.801 | RR at T0 0.064 |
| | | RR at T28 0.052 | RR at T28 0.000 | RR at T28 0.004 |
| | | No % reduction | 100% reduction | 94% reduction |
| 100 | mg/L | RR at T0 0.100 | RR at T0 4.932 | RR at T0 0.102 |
| | | RR at T28 0.09 | RR at T28 0.000 | RR at T28 0.012 |
| | | 0.56% reduction | 100% reduction | 88% reduction |
| 1000 | mg/L | RR at T0 0.468 | RR at T0 7.049 | RR at T0 0.432 |
| | | RR at T28 0.483 | RR at T28 4.344 | RR at T28 0.054 |
| | | No % reduction | 38% reduction | 88% reduction |

| | |
|---|---|
| Bromhexins | RR at T0 17.826 |
| 0.7 mg/L | RR at T28 9.542 |
| | 46% reduction |

The sucralose resulted fully non-degradable and in all the samples it was detected at the original concentration (see table S1). Differently, degradation of Bromhexine 3 resulted scarcely affected by the sample concentration and only 6%, 12% and 12% of the parent compound was still present at the end of the experiment in the 50 mg and both 100 mg/L and 1000 mg/L samples, respectively. Maltitol 2, that was fully biodegraded in the samples at 50 mg/L and 100 mg/L, was detected at 62% of the original concentration in the sample 1000 mg/L, largely contributing at the failure of the RBT at this concentration.

Furthermore, the composition of both the mixtures (A and B) at 100 mg/L and 1000 mg/L before and after 28 days under RBT conditions were studied by UHPLC-qToF analysis following the untargeted and targeted approaches.

The UHPLC-qToF method used to acquire data was characterized by a chromatographic separation in a reverse phase UHPLC column followed by qToF mass spectrometer detection. All the species eluted were selectively characterized by their retention time and high-resolution mass. Therefore, after the acquisition of the chemical fingerprint, the data were aligned. Variables with a frequency of missing value and coefficient of variation more than 20% have been eliminated, noise was subtracted, and the corresponding raw data matrix was subjected to normalization and scaling.

The resulting matrix of data was used to build the statistical model, after passing the classical test as R²2 X, Q² (model diagnostic tests), Hotelling T², DModX (observation diagnostic tests). Covariance matrix and standardized principal components were selected for unsupervised principal component analysis (PCA) computation.

The first two components of the 2D-PCA were found to comprehend more than 90% for mixture A (PC1 74.9%, PC2 15.8% 100 mg/L; PC1 57.5%, PC2 33.2% 1000 mg/L) and for mixture B (PC1 91.4%, PC2 2.2% 100 mg/L; PC1 96.1%, PC2 2.1% 1000 mg/L) of the total variance of the model, in both the experiments at 100 mg/L (FIG. 2) and 1000 mg/L (FIG. 3).

The graphics evidenced that for mixtures A the observations (score) are far from that of the mineral medium at 28 days while in the case of mixture B the biodegradation appear to be more advanced, but still not fully coincident with mineral medium (Compare FIGS. 2a/2b and 3a/3b).

Another way for a qualitative evaluation of metabolites fingerprinting obtained by UHPLC-qToF is the statistical elaboration using unsupervised “Cluster Analysis” (CA) in order to obtain the natural hierarchical groupings. By means of CA, after applying the linkage method of Ward. (H. Ward, Hierarchical Grouping to Optimize an Objective Function, J. Am. Stat. Assoc., 1953, 58, 236-244) as a standard method for the formation of the hierarchical cluster, the distance between the groups was determined using the Euclidean distance and the results of the analysis of the clusters are presented as dendrograms (FIGS. 4 and 5). The clusters generate by mixture A (at 100 mg/L) were essentially two: one formed by samples collected after 28 days under the condition of the biodegradation test and the other by the samples collected at the beginning of the study and the mineral medium (FIG. 4a). For mixture B (at 100 mg/L), similarly two clusters were identified: one is for the samples collected at the beginning of the test and the other for the samples collected after 28 days and the mineral medium, (FIG. 4b) confirming the observation of PCA. Similarly, the untargeted analysis of the samples at concentration of 1000 mg/L indicates that the degradation of mixture B produces a mixture of derivatives that are closer to that arising from the mineral medium, respect to the molecular fingerprint of the mixture obtained by the biodegradation of A (compare FIGS. 4a/4b and 5a/5b). Nevertheless, in both cases the full mineralization seems to be not completely achieved.

These data clearly indicate that considering a mixture of compounds the output of the biodegradation obtained after 28 days of RBD test is strongly affected by the chemical composition of the starting material affording new mixtures of compounds that are not fully coincident with that of the mineral medium that is supposed to be fully degradable into basic bricks such as carbon dioxide, water, ammonia, and sulphide.

Furthermore, these results demonstrate that untargeted analysis performed by UHPLC-qToF offers an interesting method to evaluate the actual biodegradation of chemical mixtures, overcoming some limitation on sensitivity showed by the currently used RBT.

These untargeted analyses provided a holistic overview of the mixture behaviour suggesting further investigations using a targeted approach, with the aim to understand the fate of each specific compounds present in the complex mixture after 28 days of RBT.

The same UHPLC-qToF technique used for the untargeted analysis, was used to characterize some compounds present in mixtures A and B. By means of this method, pure reference samples of selected compounds as sucralose, bromhexine and ambroxol (as its well-known metabolite), were analysed for their correct identification in the mixture A. The MS and MS/MS experimental data of sucralose and bromhexine were in agreement with those reported in literature. The tentative identification of compounds presents in mixture B was achieved by using a Personal Compound Database and Library (PCDL) of natural compounds present in the laboratory.

In the starting mixtures A and B, based on high resolution mass, retention time, MS/MS fragmentation patterns, isotopic profile and isotopic abundance the compounds 1-6 reported in FIG. 6 were unambiguously identified.

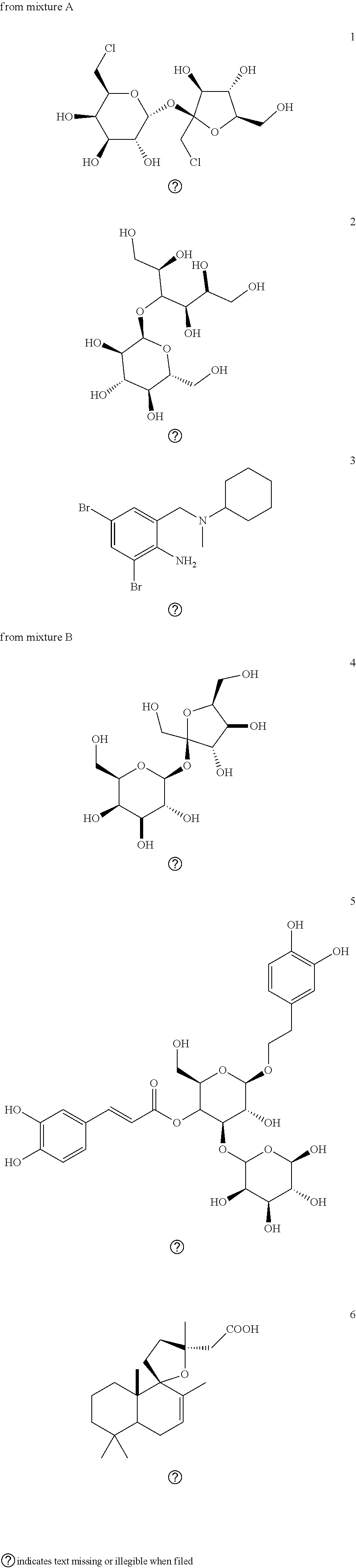

In the mixture A 1000 mg/L after 28 days under the RBT conditions UHPLC-qToF analysis was used to detect Bromhexine (3) and its metabolites. Bromhexine (3) has a typical MS/MS fragmentation characterized by the benzyl amino bond cleavage giving rise to two distinctive fragments, an aromatic one which brings the bromine atoms with a measured m/z at 263.8843, and a cyclohexane part bonded to the amino group with a measured m/z 114.1277

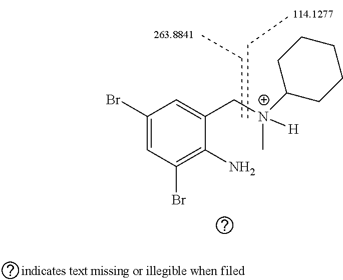 [SMILES: C[NH+](Cc1cc(Br)cc(Br)c1N)C1CCCCC1]

Molecular ion m/z 377.0048 [(M+H+2)⁺] and of the corresponding fragment ions m/z 114.1280 and m/z 263.8843 (acceptable differences below 5 ppm with the calculated m/z are observed) clearly indicates that Bromhexine and same related metabolites are still present after 28 days.

In particular the extract ion at m/z 263.8841 showed that there are several Bromhexine metabolites having as common characteristic the bromo aryl fragment. Some of them were be assigned at different level of confidence. As an example is possible to identify the structure of Ambroxol 7 (level 1) with the experimental data fully superimposable to that of the pure reference standard identified by characterized fragmentation reported below

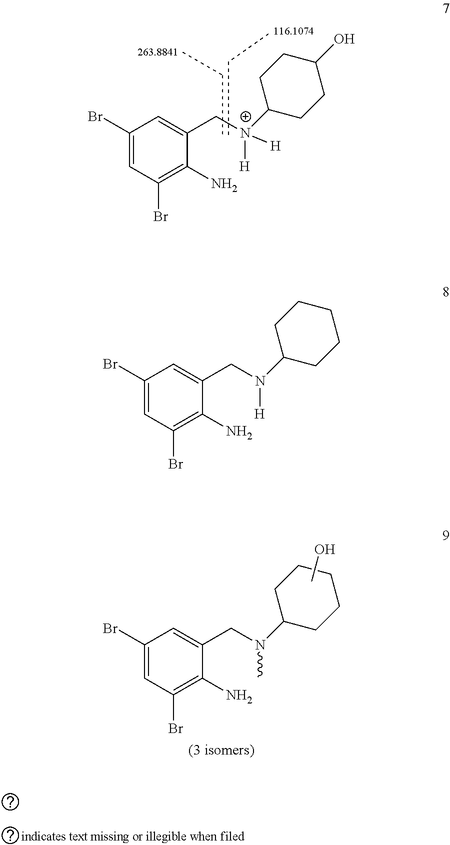

As above described from the semiquantitative analysis of the spectroscopic data emerged that 12% of the initial Bromhexine is not degraded after 28 days and that, among all, the metabolites 8 and 9 (scheme above) were the most abundant, probably because the N-demethylation of bromhexine and hydroxylation of the cyclohexyl ring were reactions kinetically favourable for the metabolism of RBT microfauna.

Similarly on mixture A it was performed the target analysis of sucralose. Searching for the molecular ion m/z 419.0038 [(M+Na)⁺] after 28 days its presence has been detected without any significant difference in concentration. This result was further confirmed by the EIC of its fragment at m/z 238.9848, that evidenced that in this case the presence of correlable metabolites having a common fragmentation was not detectable.

The target study on biodegradability of the mixture B was performed focusing the attention on the fate of Sucrose (4), Acteoside (5), Grindelic acid (6) as selected representative compounds of the mixture, identified at the T0 of the RBT.

The experimental MS/MS fragmentation pattern of Acteoside (5) and Grindelic acid (6) have been schematized below:

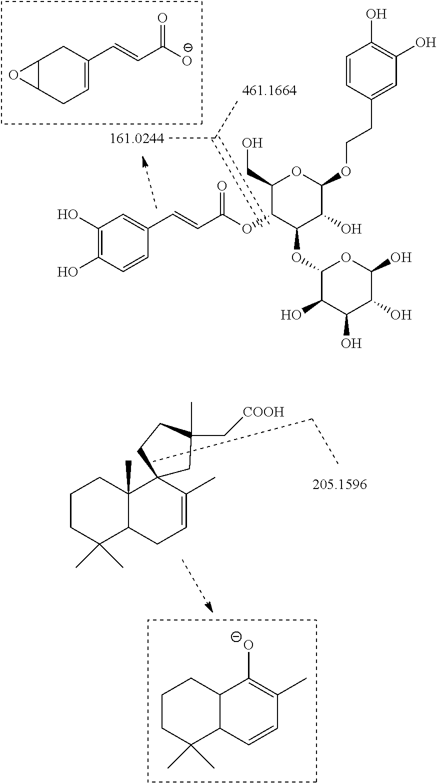

Acteoside showed the loss of the caffeoyl moiety corresponding to m/z 161.0244 [caffeic acid-H₂O—H⁻], giving rise to the anion m/z 461.1664 obtained by difference [M−H-caffeoyl⁻]. Grindelic acid (6), gave rise to the mass fragment 205.1596, assuming a spiro-tetrahydrofurane ring opening and further anion rearrangement not jet reported in literature. The Elemental composition of the proposed fragment is C₁₄H₂₁O, with a calculated monoisotopic exact mass at m/z 205.1596, which correspond to the m/z recorded from the fragmentation of Grindelic acid pure standard as well in the sample.

It was not possible to detect the presence of compounds 4, 5 and 6, neither of their direct derivatives after 28 days of RBT.

Finally, in order to compare the fate of a not fully degradable API as a pure chemical entity or as a part of a pharmaceutical formulation he RTB test of pure Bromhexine (3) was performed at 0.7 mg/L corresponding to its concentration in the mixture A 1000 mg/L. After 28 days the evaluation of the relative area % (Table S1) analysis evidenced that only 46% of the pure compound was degraded (vs 88% observed in the mixture A). This result is not easy to interpretate and can be tentatively attributed to a higher activity of the microfauna in the presence of other readily available sources of energy. However, it clearly indicates that the degradation of a specific molecule can be considerably different when obtained in its pure form or as a part of a complex chemical mixture, suggesting that the mixture, similarly to a complex system, can exploit some emerging and non-fully predictable properties. In conclusion based on the results collected in this manuscript we propose that the use of modern techniques, (such as UHPLC-qToF) should be considered for a more accurate and reliable evaluation of the biodegradation and, consequently, the environmental impact of pharmaceutical and cosmetic formulations. We here demonstrated that using an untargeted approach, two different pharmaceutical formulations that, according to the currently used analytical protocol, can be considered readily biodegradable, are indeed not fully mineralized neither considering the synthetic components nor the natural ones even if, as expected, this latter showed a much more advanced degradation toward the mineral medium. The uncomplete mineralization of a formulation affords a new mixture of derivatives that may deserve to be analysed in detail using highly performant methods like those based on mass spectrometry. Furthermore, we also observed that the biodegradability of a mixture of compounds cannot be considered as the simple sum of the biodegradability of each component. In this direction the new technologies offer a holistic approach able to consider all at once the fate of the chemical system that have to be biodegraded and, at the same time, a very sensible target analysis able to elucidate most of the component of the final chemical fingerprint.

For these reasons, in our opinion, the use of these modern technologies and the development of new protocols and rules based on it, will should improve the ability on predict, control and prevent the environmental impact of chemical mixtures as that present on pharmaceutical, cosmetic, and personal care products market.

The results here reported suggest a new approach in the evaluation of biodegradability, more based on systemic thinking, a paradigm that must always be considered when talking about the environment and biological systems.

### Mixtures C and D

Two commercially available complex formulated products commonly used for the treatment of Reflux disease and functional dispepsia, one containing synthetic ingredients (Mixture C) and one containing only natural ingredients (Mixture D), were chosen as real examples of largely distributed formulations of pharmaceutical ingredients to study transformation products during Ready Biodegradability Tests.

The ingredients of the two products, declared in the package leaflet, are summarized in Table 1 above, being the “Mixture C” based on Omeprazole. Omeprazole is currently one of the pharmaceutical active ingredients most widely presents in the world as well in Italy-according to the latest report by the Italian Medicines Agency—in drugs consumed for the treatment of gastric diseases.

### Ready Biodegradation Test

The respirometric-manometric test OECD n° 301F [OECD, «Revised Introduction to the OECD Guidelines for Testing of Chemicals, Section 3, OECD» 2006, OECD, Test No. 301: Ready Biodegradability, 1992.] was chosen as method for the ready biodegradability evaluation of our two complex formulations. Each experiment was set up according to OECD guidelines, thus the inoculum obtained from the withdrawal of activated sludge and river water in equal rates and characterized in its macro constituents (as defined experimental part) was used. The inoculum wasn't preadapted to the mixtures tested, thus the results can be considered representative and robust. The oxygen consumption was recorded by means of a calibrated continuous reader probe. To be sure of the repeatability of the results for each product three replicas were performed. At a concentration of 50 mg/L “Mixture C” resulted not readily biodegradable, because after having eliminated the contribution of nitrification at 28th day resulted a 26% of biodegradation. “Mixture D”, tested at the same concentration, resulted readily biodegradable as passed the ten-days window criteria and reached the 91% of biodegradation at 28th day (Table 3).

| | CONCEN- | BIODEGRADATION | |
|---|---|---|---|
| PRODUCT | TRATION | AT 28^{TH} DAY¹ | RESULT |
| MIXTURE C | 50 mg/L | 26% | Not Readily |
| “OMEPRAZOLE | | | Biodegradable |
| BASED” | | | |
| MIXTURE D | 50 mg/L | 91% | Readily |
| | | | Biodegradable |

¹The pass level for ready biodegradability TEST WITH respirometric method is the achievement of 60% of ThOD removal. This pass value has to be reached in a 10-d window within the 28-d period of the test. The 10-d window begins when the degree of biodegradation has reached 10% ThOD and must end before day 28 of the test. Chemicals which do not reach the pass levels are not considered readily biodegradable

For “Mixture D” the test was repeated at the concentrations of 75 mg/L, confirming the results obtained at 50 mg/L.

The transformed products of “mixture C” at 50 mg/L and “Mixture D” 75 mg/L after 28 days under OECD 301F test conditions were studied by UHPLC-qToF analysis following untargeted and suspect screening approaches.

### Sample Preparation

Targeted suspect screening and untargeted analysis workflows involve multiple steps ranging from sample preparation and data acquisition to data mining, expert reviewing and data interpretation. The first sample preparation step is critical and a compromise between selectivity and sensitivity has to be sought.

The concept of cleaning to remove most abundant interferences was born on procedures used for targeted methods, to enrich low concentrated analytes by purification. But it generates new issues, especially when the nature of the compounds to be detected are not fully known in advance.

In the context of our research, sample handling was minimized to be as non-selective as possible with respect to the various transformation compounds. Therefore, the test mixtures were simply subjected to a filtration step, with the advantage of preserving the integrity of the sample, thereby limiting the variability related to sample preparation.

### UHPLC-ESI-qToF Analysis

The UHPLC-qToF method used to acquire fingerprint data was characterized by a chromatographic separation in a reverse phase UHPLC C18 column followed by qToF mass spectrometer detection. The ESI source was used to produce the various ion molecular species. The positive ion mode was used to analyze “Mixture C”, due to the presence of the nitrogenous compound “Omeprazole”. While the ionization in negative ion mode was applied in the case of “Mixture D”, as it was rich in phenolic compounds. The mobile phase was composed of water/methanol according to a generic elution gradient, from a high percentage of water to a high percentage of organic solvent, thus satisfy the separation of most of the analytes and limiting the matrix effects. A final wash of the column (e.g. with 99% of methanol) was present, to avoid carry-over between injections. Formic acid as phase modifier was added to the mobile phase to stabilize the pH, increase peak shape and promote ionization.

To ensure the repeatability and reach a better level of confidence on the produced results each sample was acquired in triplicates and pooled samples were collected at the beginning, middle and end of the analytical session.

### Data Processing

The post-acquisition data processing step consists of shifting from raw instrumental data to a curated tabulated file containing the list of peaks used for subsequent annotation and statistical analyses for targeted suspect screening and untargeted analysis, respectively. This part fundamentally depends on the availability and performance of bioinformatics tools. In our case, to perform the extraction of information from the raw data Mass Profiler Professional (from Agilent), R package (from R foundation) and Simca (from Umetrics/Sartorius) were used.

### Untargeted Analysis

Basically, untargeted analysis was performed with the aim to detect any signal present in the generated chemical fingerprint from the readily biodegradation test and visualize the test results after unsupervised multivariate statistical analysis.

Therefore, peak picking, peak alignment and peak integration were performed, respectively, to align common peaks found in the different samples and report their intensity or area. Variables with a frequency of missing value and coefficient of variation more than 20% have been eliminated, noise was subtracted, and the corresponding raw data matrix was subjected to normalization and scaling.

The resulted peak list is characterized with a series of features labelled with retention time and HR-MS. The so obtained matrix of data was used to build the statistical model, after posing the classical test R²X, Q² (model diagnostic tests), Hotelling T² and DModX (observation diagnostic tests) at 95% of confidence level. Covariance matrix and standardized principal components were selected for unsupervised principal component analysis (PCA) computation. How is possible to observe, in the 2D diagrams (FIG. 7) the first two components of the 2D-PCA were found to comprehend more than 95% for “Mixture C” (PC1 70.7%, PC2 26.9%) and for “Mixture D” (PC1 98.3%, PC2 0.6%) of the total variance of the model.

The graphics evidenced that for “Mixture C” the observations (score) are far from that of the mineral medium at 28th day while in the case of “Mixture D” the biodegradation appear to be quite fully coincident with mineral medium.

Another way for the qualitative evaluation of the fingerprinting obtained by UHPLC-qToF is the statistical processing using unsupervised “Cluster Analysis” (CA) in order to obtain the natural hierarchical groupings. By means of CA, after applying the linkage method of Ward [H. Ward, Hierarchical Grouping to Optimize an Objective Function, J. Am. Stat. Assoc., 1953, 58, 236-244] as a standard method for the formation of the hierarchical cluster, the distance between the groups was determined using the Euclidean distance and the results of the analysis of the clusters are presented as dendrograms.

The clusters generate by “Mixture C” were essentially three: one formed by samples collected after 28 days under the condition of the biodegradation test, another by the samples collected at the beginning of the study and another by the mineral medium (FIG. 3). For “Mixture D”, two clusters were identified: one is for the samples collected at the beginning of the test and the other for the samples collected after 28 days and the mineral medium, (confirming the observation of PCA).

### Targeted Suspect Screening Analysis

Using the targeted suspects screening analysis, the annotation of the suspect compound is obtained with various level of confidence, depending on the type and extent of structural information collected and available through the analytical workflow. In the present case, the raw data collected at the beginning of the RBT and on day 28 were background subtracted and matched with the data present in two compounds library:
the “in-house PCDL” experimental database containing a thousand of natural compounds characterized by their retention time, accurate mass and accurate MS/MS fragments
the Metlin_Metabolites_AM_PCDL, a database containing accurate mass and accurate MS/MS fragments for a wide range of compounds and metabolites.

Both the libraries were integrated into Mass Hunter qualitative analysis (Agilent) and were used to achieve identification of natural compounds and Omeprazole TPs using “find by formula” mining algorithm. Obviously the “Metlin PCDL” was specific for “Mixture C” and the “in-house PCDL” was specific for “Mixture D”.

At the beginning of the test, in the mixture of “Mixture C” some typical compounds as sucrose, liquiritin and apigenin were detected, while Omeprazole was detected in the mixture of “Mixture D”. The presence of these compounds agrees with the composition of the two pharmaceutical products. Then their high-resolution mass, retention time, high resolution MS/MS fragmentation patterns, isotopic spacing and isotopic abundance were in agreement with data from certified reference standards.

The same search on the two libraries was carried on the 28th day. Form the evaluation of the data emerged that Omeprazole in “Mixture C” was still detected, while in “Mixture D” sucrose, liquiritin and apigenin were no more detectable. The data for Omeprazole is in agreement with that already known.

### Semi-Quantitative Determination

From the semi-quantitative determination by area % evaluation, among all the Omeprazole TPs at t28 the most abundant appeared Omeprazole TP_12 (Omeprazole TP_12 5-methoxy-2-((4-methoxy-3,5-dimethylpyridin-2-yl)methylthio)-1H-bendo[d]imidazole (ufiprazole), followed by TPs 5, 7 (Omeprazole TP_5 4-methoxy-3,5-dimethyl-picolinic acid (CAS n° 138569-60-5; Omeprazole TP_7 5-Methoxy-2-(4-methoxy-3,5-dimethyl-methylenepyridin-1 (2H)-yl)-1H-benzo[d]imidazole) and the others.

It can be deduced that after 28 days a series of Omeprazole transformation compounds were formed, demonstrating that the metabolic rate was kinetically unfavourable for the microfauna of the OECD 301F ready biodegradability test.

We tested the ready biodegradability of “Mixture C”-Omeprazole based- and “Mixture D” applying the respirometric-manometric test (OECD 301F).

“Mixture C” resulted not readily biodegradable, while “Mixture D” was readily biodegradable.

Samples from readily biodegradability test were studied applying UHPLC-qToF “all-ion MS/MS” technique to perform untargeted and suspect screening analysis to follow the behaviour of two antiacid pharmaceutical products.

Significant changes in the samples from the beginning of the respirometric-manometric test to the 28th day were observed, as a result of the consumption of the products by the microfauna. The incomplete mineralization was observed for “Mixture C” was also proven by the untargeted analysis the suspect screening study. On the other hand, for “Mixture D” the selected compounds were note detected, being better metabolized by the microfauna.

In conclusion, these data highlighted that the use of UHPLC-qToF “all-ion MS/MS” methods permits several analytical approaches has a great potential in the future of biodegradability studies to evaluate the behaviour of compounds even when they are present in complex mixtures.

### Materials and Methods

### Biodegradation Method

Magnesium sulphate heptahydrate for analysis ACS Reag. Ph.Eur and Reag. USP, sodium acetate anhydrous USP, Phosphoric acid 99%, Potassium phosphate dibasic 99+% anhydrous for analysis, Potassium phosphate monobasic 99% anhydrous for analysis, Iron (III) chloride hexahydrate Pure, Sodium phosphate dibasic dihydrate 98+%, calcium chloride anhydrous 99% pure, were purchased from Carlo Erba Reagents srl (Cornaredo, Milano, Italy). Ammonium Chloride 99% puriss and potassium hydroxide 85% puriss were purchased from Italchimica SPA (Pontecchio Polesine, Rovigo, Italy). Purified water was prepared by Arium Mini water purification system (from Sartorius Goettingen, Germany).

Sample Preparation: The sample was treated according to the procedure reported in the respirometric manometric method n°301F (OECD). To be able to have the correct registration by the BOD sensor and enough material to perform the instrumental analysis planned in a 1 L vessel 0.4 L of mineral medium and 50 mg/L or 75 mg/L of sample were introduced. Sodium acetate was used as reference substance. Tests were done at the constant temperature on 22° C.

Inoculum preparation. The inoculum was prepared collecting 5 samples of activated sludge and 5 samples of water river from different places and mixed in equivalent volume. the inoculum is oxygenated, stirred and fed with glucose, peptone, potassium phosphate dibasic and the values of redox potential, oxygen consumption and total dry matter are monitored daily. At the time of use, the dry substance was determined at 100° C. to measure the same quantity (30 mg/ml) in the vessels containing the test substance.

Inoculum composition. The inoculum was obtained by mixing active sludge from 8 different sectors and river water taken from two different rivers in equal parts. The assembled inoculum was oxygenated, stirred and fed with glucose, peptone and monopotassium orthophosphate. Oxygen, redox, and total suspended solids values were monitored daily.

Before the use of inoculum, the total dry matter is determined.

The composition of the Microfauna was determined by optical microscope analysis and was composed by the following species: Litonotus Fasciola, Aspidisca costata, Vorticella acquadulcis, Vorticella convallaria, Amoebas, Rotifers, Tardigrades.

Instrumental parameters. The biological oxygen demand was constantly monitored by means of BOD EVO Sensor System 6, being made up of six BOD EVO sensors, a six-position stirring base, dark glass bottles, sub-caps for the absorption of carbon dioxide and magnetic stir bars. The BOD EVO sensor transmits wireless the data to the dedicated BODSoft™ software (VELP Scientifica, Usmate, Monza-Brianza, Italy). All the experiments were conducted under controlled temperature in a refrigerated thermostat provided with auto-tuning thermoregulation system and forced air circulation in order to have stability and homogeneity of the internal temperature (VELP Scientifica, Usmate, Monza-Brianza, Italy). The temperature of the thermostat was monitored by means of a datalogger with external probe with a range of temperature from −40 to +80° C. (XS Instruments, Carpi, Modena, Italy).

### UHPLC ESI-qToF Methods

All solvents were of high purity analytical grade and were used without further purification. ULC/MS grade absolute methanol was purchased from Biosolve (Dieuze, France). Ultrahigh purified water was prepared in a PURELAB® Ultra water purification system (ELGA, UK). Formic acid 98%-100% for LC-MS LiChropur® and dimethyl sulfoxide ≥99% were purchased from Sigma-Aldrich (St. Louis, MO).

“Mixture C” containing Omeprazole was purchased (at a local pharmacy, batch n° LC52792), “Mixture D” was produced by Aboca (batch n° 21D1848); Omeprazole was purchased from Sigma-Aldrich (St. Louis, MO).

The high-purity reference standards, used both for the construction of the in-house database and for the construction of the calibration curves, were purchased from Extrasynthese (Genay, France), Sigma-Aldrich (St. Louis, MO), PhytoLab GmbH & Co. KG (Vestenbergsgreuth, Germany) and ChromaDex (Irvine, CA). High-purity reference standards stock solutions were prepared in methanol, water/methanol (80:20, v/v) or methanol/dimethyl sulfoxide (80:20, v/v) at 500 ppm. The working solutions were prepared by diluting appropriate volumes of the stock solutions with water/methanol (50:50, v/v). Internal standard sulfadimethoxine-d6 was purchased from Sigma-Aldrich (St. Louis, MO). Internal standard stock solution was prepared in methanol at 5 mg/L. All the stock solutions were stored in glass vials at −80° C.

Each sample subjected to fingerprint is filtrated before acquiring said fingerprint in order to eliminate the microfauna thereby substantially stopping the biodegradation process at the desired time t.

### Targeted Suspect Screening Method

Sample preparation: The sample was filtered on a 0.20 μm Millipore cellulose acetate syringe filter and was used for the acquisition of the chromatographic profile without any dilution according to the following conditions.

Instrumental parameters. The instrumental platform used consisted of an UHPLC series 1290 coupled to a quadrupole time-of-flight (qToF) mass spectrometer series 6545 (Agilent Technologies, Santa Clara, CA) in high resolution (2 GHZ). The UHPLC was endowed with a binary pump, an autosampler, a multicolumn thermostat, an isocratic pump and a solvent cabinet. The autosampler was maintained at 15° C.

For “Mixture C” the analytical column was a ACQUITY UPLC BEHR C18 (100×2.1 mm, 1.6 μm) protected by a ACQUITY UPLC BEH UPLC® C18 VanGuard™ pre-column (5×2.1 mm, 1.6 μm) both supplied by Waters (Milford, MA) and thermostated at 40° C.

For “Mixture D” the analytical column was a Cortecs® C18 (100×2.1 mm, 1.6 μm) protected by a Cortecs® UPLC® C18 VanGuard™ pre-column (5×2.1 mm, 1.6 μm) both supplied by Waters (Milford, MA) and thermostated at 40° C.

The analyses were performed in elution gradient using aqueous HCO₂H (0.1%, v/v) as mobile phase A and HCO₂H (0.1%, v/v in MeOH) as mobile phase B according to the following gradient (Table 7), with a flow rate of 0.3 mL min-1. The volume injected was 3 μL for the samples at 1000 mg/L, while was 10 μL for the samples at 100 mg/L.

TABLE 4
Elution gradient.

| Time (min) | A % | B % |
|---|---|---|
| 0.0 | 99 | 1 |
| 1.0 | 99 | 1 |
| 2.0 | 75 | 25 |
| 10.0 | 50 | 50 |
| 11.0 | 50 | 50 |
| 15.0 | 25 | 75 |
| 17.0 | 15 | 85 |
| 19.0 | 1 | 99 |
| 19.5 | 1 | 99 |
| 21.0 | 99 | 1 |
| 24.0 | 99 | 1 |

The UHPLC system was coupled to a qToF mass spectrometer endowed with a Dual AJS ESI source operating in positive ionization mode for “Mixture C” and in negative ionization mode for “Mixture D” with a scan range from 50 m/z to 1700 m/z. The optimized instrument parameters are reported in Table 5.

TABLE 5
MS parameters

| Instrumental parameters | ESI− | ESI+ |
|---|---|---|
| Gas temperature (° C.) | 325 | 325 |
| Gas flow (L min−1) | 11 | 11 |
| Nebulizer (psig) | 35 | 35 |
| Sheat gas temperature (° C.) | 350 | 350 |
| Sheat gas flow (L min−1) | 12 | 12 |
| VCap | 3500 | 4000 |
| Nozzle voltage (V) | 0 | 1500 |
| Fragmentor | 100 | 100 |
| Skimmer | 65 | 65 |
| Octopole RF Peak | 750 | 750 |

The construction of the in-house PCDL was performed by acquiring each analytical standard reference compound in data dependent mode (Auto MS/MS, Agilent Technologies) using three different collision energy values (20, 30 and 40 eV) by N2. While the acquisition of the sample fingerprint under investigation was performed in All-Ions mode using 30 eV of collision energy value. In both the acquisition methods, a reference mass solution containing purine and hexakis (1H, 1H,3H-tetrafluoropropoxy) phosphazine was direct injected to the ESI source by the isocratic pump and ionized together with the sample solution for mass correction allowing to get accurate mass time-of-flight data.

Software. MassHunter software version B.07 (Agilent Technologies, Santa Clara, CA) was used for data acquisition and processing. The exact mass of the various molecular species was calculated by means of Isotope distribution calculator a tool of MassHunter Data Analysis core, version 8.0.8208.0 (Agilent Technologies, Santa Clara, CA). The Metlin library used was Metlin_Metabolites_AM_PCDL, version 7.0.

Data Analysis. Elaborations were performed by means of “Qualitative analysis” program and by “Find-by-Formula” algorithm. The data file is loaded into “Qualitative analysis” then “Find-by-Formula” is run on the low channel against an MS/MS library. Find-by-Formula returns possible precursor formulas found as well as their product ions in the library. Using the list of product ions, “Qualitative analysis” extracts EICs from the high channel and aligns them with an EIC of the precursor. A coelution score is calculated and compounds which pass the threshold (user-set at 70) are retained.

“Qualitative analysis” returns the overall score to which contribute: the mass score, the isotope abundance score, the isotope spacing score and the retention time score the threshold was user-set at 70. EICs were taken by setting m/z extension to symmetric 2.5 ppm.

### Untargeted Method

Sample preparation: Samples were prepared as described for the targeted suspect screening method.

### Multivariate Statistical Analysis

Data collection. Samples of the mixture Ct T0 and T28 and the blank sample, consisting of mineral medium and inoculum, were organized into a randomized sample queue prior to UHPLC-qToF acquisition. The analytical blank and pooled samples were acquired at the beginning, in the middle of and at the end of the sample sequence. Each sample was acquired in triplicate. Instrumental parameters were the same used for the suspect screening, positive ions for “Product” A and negative ions for “Mixture D”

Data processing. Peak peaking and peak alignment were obtained using the software R [R version 3.5.2 (2018 Dec. 20), Copyright (C) 2018 The R Foundation for Statistical Computing]. Mass tolerance was set at 10 ppm and retention time tolerance was set at 10 seconds. Signal Filtering Threshold was set at 1000 counts. Variables with a frequency of missing value and coefficient of variation more than 20% have been eliminated and data imputation was performed. Additionally, a filter to eliminate those variables present in the analytical blank samples was applied.

Median of the replicates was applied to remove the effect of the random noise, while data normalization was performed by centering and scaling with PQN (Probabilistic Quotient Normalization) on reference pooled samples.

The processed data were saved in CSV format and the data matrix in CSV format has been reworked by the program SIMCA (Version 16.0.2.10561, Jan. 22, 2020, Umetrics/Sartorius). Later On, data matrix was mean centered and Pareto scaling was applied prior to perform data analysis.

Statistical Model performance evaluation was done and a confidence level of 95% was used. The goodness of fit and the goodness of prediction were evaluated by means of Q²X and R²X, respectively.

| | Q² | R² |
|---|---|---|
| “Mixture C”_50 mg/L (Omeprazole) | 0.948 | 0.985 |
| “Mixture D”_75 mg/L | 0.795 | 0.989 |

The diagnostic tools Hotelling T² and dModX were calculated to be sure that the model is not deformed therefore neither strong nor weak outliers were present. The models were robust, as the observations were below the calculated critical values and there were no outliers.

PCA analysis. After data processing, in order to evaluate the complex fingerprints obtained, multivariate analysis of the Principal Component (PCA) was used. The PCA model was built using the first two principal components providing a 2D model (Confidence level 95%).

Cluster Analysis. Cluster analysis was performed according to an unsupervised approach using a hierarchical algorithm (Confidence level 95%). Distance indices were determined using Ward's linkage method and Euclidean distance.

Semiquantitative determination. At T28, the Omeprazole area counts and that of the compounds identified were divided by the internal standard (sulfadimethoxine-d6) area count. Then all the corrected values are added together to get a total area. The percentage of the area of each compound at T28 is calculated by dividing each corrected area by the total area and multiplied by 100.

1. A method for the assessment of the biodegradability of mixtures of organic compounds comprising the following steps:
a) preparing at least one test flask or vessel containing a predetermined amount of a mixture of organic compounds of interest suspended in a suitable mineral medium together with an inoculum wherein said inoculum is obtained from activated sludge; sewage effluents (unchlorinated); surface waters and soils; or from a mixture thereof and at least one blank flask or vessel containing only said mineral medium and said inoculum;
b) acquiring the fingerprint by liquid chromatography coupled to mass spectrometry of a sample from each flask or vessel prepared in a) at T0, T0 being the day of the preparation a) wherein each sample is submitted to filtration in order to eliminate microfauna therefrom before acquiring said fingerprint;
c) acquiring the fingerprint by liquid chromatography coupled to mass spectrometry of a sample from each test flask or vessel prepared in a) at Tn, n being any integer >0 and representing the number of the day after said preparation in a) wherein each sample is submitted to filtration in order to eliminate microfauna therefrom before acquiring said fingerprint;
d) carrying out a multivariate statistical analysis on the data obtained in b) and c); and
e) comparing the multivariate statistical analysis results obtained in d) for each test sample and for the blank sample and assessing the biodegradation of each test sample by evaluating the distance between the data obtained for the blank sample at Tn and for the test samples at least at T0 and at said Tn.

2. The method of claim 1 wherein said step c) is repeated at different Tn and said steps d) and e) are carried out for each fingerprint of the test sample and blank sample acquired at the same Tn for each value of n.

3. The method of claim 1 wherein one of said Tn is T28, T28 being the 28^{th} day after said preparation a).

4. The method of claim 1 further comprising
f) measuring oxygen consumption, and/or carbon dioxide production, and/or dissolved organic carbon consumption by means of calibrated continuous reader probe/s, or by making discontinuous readings by arranging an appropriate number of vessels for each scheduled measurement, from T0 to T28.

5. The method of claim 1 wherein said mineral medium is prepared from stock solutions of appropriate concentrations of mineral components potassium and sodium phosphates plus ammonium chloride, calcium chloride, magnesium sulphate and iron (III) chloride.

6. The method of claim 1 wherein said medium is further supplemented with suitable growth factors.

7. The method of claim 1 wherein said test samples are organised in a randomised sample queue prior to liquid chromatography coupled to mass spectrometry acquisition wherein said sample queue also comprises one or more pooled sample and one or more blank sample.

8. The method of claim 1 wherein said liquid chromatography coupled to mass spectrometry is carried out by UHPLC-qToF.

9. The method of claim 1 wherein said multivariate statistical analysis is an untargeted unsupervised analysis.

10. The method of claim 1 wherein said multivariate statistical analysis is carried out by PCA (principal component analysis) and/or HCA (hierarchical cluster analysis).

11. The method of claim 10 wherein said multivariate statistical analysis is carried out by PCA and in step e) when the PCA results of the test sample at Tn are near the 2D space of the PCA results of the blank sample at the same Tn, the test sample is considered to be biodegraded and when the PCA results of the test sample at Tn are not near the 2D space of the PCA results of the blank sample at the same In the test sample is considered to be not biodegraded or only partially biodegraded and/or
wherein said multivariate statistical analysis is carried out by HCA and in step e) when the HCA results of the test sample at Tn form a cluster with the HCA results of the blank sample at the same Tn, the test sample is considered to be biodegraded and when the HCA results of the test sample at Tn do not form a cluster with the HCA results of the blank sample at the same Tn, the test sample is considered to be non-biodegraded or only partially biodegraded.

12. The method of claim 1 wherein a targeted qualitative analysis of one or more of the test samples is carried out.

13. The method of claim 1 wherein said mixture of organic compounds is a pharmaceutical composition or a cosmetical composition or a food supplement composition or a medical device composition or a nutraceutical composition.
